(19) United States
(12) Patent Application Publication
(10) Pub. No.: US 2021/0052882 A1
(43) Pub. Date: Feb. 25, 2021

(54) ENHANCED TREATMENT VOLUME AND SELECTIVE ABLATION USING ELECTROPORATION WITH ADJUVANT CALCIUM

(71) Applicant: Virginia Tech Intellectual Properties, Inc., Blacksburg, VA (US)

(72) Inventors: Elisa M. Wasson, Blacksburg, VA (US); Rafael V. Davalos, Blacksburg, VA (US)

(21) Appl. No.: 17/000,049

(22) Filed: Aug. 21, 2020

Related U.S. Application Data
(60) Provisional application No. 62/889,842, filed on Aug. 21, 2019.

(51) Int. Cl. A61N 1/04; A61B 18/14

(52) U.S. Cl. CPC A61N 1/0412; A61B 2018/00613; A61B 18/1477

(57) ABSTRACT

High-frequency irreversible electroporation (H-FIRE) is an electroporation-based therapy used to ablate cancerous tissue. Treatment consists of delivering short pulses in a series of bursts. Reducing pulse duration leads to reduced treatment volumes compared to traditional IRE, therefore larger voltages are typically applied to generate ablations comparable in size. Administration of adjuvant calcium enhances ablation area in vitro for H-FIRE treatments of several pulse durations. Furthermore, H-FIRE treatment delivered with CaCl₂ results in cell death thresholds higher than that of H-FIRE without calcium and comparable to IRE thresholds without calcium. Quantifying the reversible electroporation threshold revealed that CaCl₂ enhances the permeabilization of cells compared to a NaCl control. H-FIRE treatment with calcium enhances the IRE to thermal cell death ratio, thereby also enhancing the positive immune response from treatment.

### CROSS-REFERENCE TO RELATED APPLICATIONS

The present application relies on the disclosure of and claims priority to and the benefit of the filing date of U.S. Provisional Patent Application No. 62/889,842, filed Aug. 21, 2019, which is hereby incorporated by reference herein in its entirety. Additionally, the present application is related to U.S. Pat. Nos. 8,465,484, 8,814,860, 8,926,606, 8,992,517, 9,198,733, 9,283,051, 9,598,691, 9,867,652, 10,117,707, 10,154,874, 10,238,447, 10,245,098, 10,245,105, 10,272,178, 10,286,108, 10,292,755, 10,448,989, 10,470,822, 10,471,254, 10,537,379, and 10,694,972; U.S. Patent Publication Nos. 2013/0184702, 2015/0289923, 2019/0029749, 2019/0069945, 2020/0093541, 2019/0133671, 2019/0175248, 2019/0175260, 2019/0223938, 2019/0232048, 2019/0233809, 2019/0256839, 2019/0282294, 2019/0328445, 2019/0351224, 2019/0376055, 2020/0046432, 2020/0093541, and 2020/0197073; International Patent Publication Nos. WO2009/134876, WO2010/118387, WO2010/151277, WO2011/047387, WO2012/0088149, WO2012/071526, WO2015/175570, and WO2020/061192; U.S. patent application Ser. Nos. 13/958,152, 16/865,031, 16/865,772, 16/915,760, and 16/938,778 each of which is incorporated by reference herein in its entirety.

### BACKGROUND OF THE INVENTION

### Field of the Invention

The present invention relates to the field of medical therapies involving administering electrical treatment energy. Embodiments of the invention provide electrical energy based methods for (i) selectively enhancing ablation in a tissue region, such as by increasing treatment margins, e.g., without increasing voltage, (ii) selectively protecting a tissue region from effects of ablation, e.g., to reduce thermal damage, spare healthy or critical structures from damage, and/or allow for an enhanced reversible electroporation zone (such as for intracellular delivery of drugs across the blood-brain barrier) and/or enhanced electrochemotherapy, (iii) enhancing the IRE to thermal cell death ratio, and/or (iv) providing a more positive/effective immune response. Embodiments of the invention in particular are useful for treating tumors, especially cancerous tumors.

### Description of Related Art

Every mammalian cell has a plasma membrane that serves to maintain homeostasis by strictly regulating the flux of ions and molecules into and out of the cell. As a result of this ion balance, cells have a transmembrane potential (TMP), which at a resting state, is around −70 mV. In the presence of an electric field however, the TMP will rise. With a sufficiently high electric field, the lipid bilayer of the plasma membrane begins to form nano pores in the membrane in a process known as electroporation. These pores allow exchange of molecules and ions, resulting in a loss of homeostasis. If the TMP reaches a critical value (˜1 V), the membrane is damaged to an extent that the cell cannot recover, hence introducing irreversible electroporation (IRE).

IRE was first demonstrated for the use of tumor ablation by Davalos et al. (Davalos, R. V., Mir, L. M., and Rubinsky, B., Ann. Biomed. Eng., 2005, 33, 223-231) and has since been used to treat unresectable tumors in human patients. In a typical IRE treatment, two electrodes are inserted directly into the tumor and 80 monopolar, square-wave pulses that are 100 μs in duration are delivered at an electric field strength of 1-2 kV/cm (voltage-to-distance ratio) and frequency of one pulse per second. A generic unipolar pulse scheme is provided in FIG. 1A. During an IRE treatment, there are four zones of electroporation: 1) a small zone of cell death caused by thermal damage (Joule heating), 2) a medium sized zone of necrotic tissue in which cells are electroporated, lose homeostasis and are unable to recover, 3) a large zone of apoptotic cell death in which defects in the membrane close, but cells are unable to recover from a loss of homeostasis, and 4) a zone of reversibly electroporated cells that recover and survive. (Lee, R. C., D. J. Canaday, and S. M. Hammer. Transient and stable ionic permeabilization of isolated skeletal muscle cells after electrical shock. J. Burn Care Rehabil. 14:528-540, 1993.) It has been found that the use of short pulses results in minimal thermal damage (Davalos, R. V., and Rubinsky, B., Int. J. Heat Mass Transf., 2008, 51, 5617-5622), unlike other ablation modalities that rely on tissue heating such as microwave and radiofrequency ablation. IRE has also been shown to spare critical structures such as blood vessels and nerves (Li, W., Fan, Q., Ji, Z., Qiu, X., and Li, Z., PLoS One, 2011, 6, e18831.; Rossmeisl, J. H., Garcia, P. A., Pancotto, T. E., Robertson, J. L., Henao-Guerrero, Neal, R. E., Ellis, T. L., and Davalos, R. V., J. Neurosurg., 2015, 123, 1008-1025).

Although IRE has shown promise in the treatment of difficult to treat tumors such as glioblastoma, it comes with some challenges. Since the pulses are monopolar and relatively long, they may induce muscle contractions and typically involve the use of neuroparalytic agents to minimize the effects. IRE is also highly dependent on cell size and tissue geometry, making it difficult to predict treatment outcomes in heterogeneous tissue.

High Frequency IRE (“H-FIRE”) has been shown to depend less on cell size and tissue geometry as well as mitigate muscle contractions and uses bipolar square wave pulses with durations of 1-10 μs, delivered in a rapid burst. Since H-FIRE individual pulse widths are much shorter than IRE individual pulse widths, the cell is permeabilized to a lesser degree than with IRE treatment and larger applied voltages are typically required to induce an equivalent lesion. With larger applied voltages (typically 2-3 kV/cm), however, comes the risk of inducing thermal damage and muscle contractions. Applying a higher energy treatment can negate advantages of HFIRE because it may introduce deleterious thermal damage, as well as increase the changes to producing muscle contractions therefore increasing the potential of needing a neuromuscular block, and/or contribute to reducing, impeding or otherwise not promoting a positive immune response.

Reversible electroporation can be achieved using lower voltage pulse parameters that cause permeabilization of the membrane, allowing molecules to enter the cell, while permitting subsequent recovery. This technique has been used in gene transfection, blood-brain barrier disruption, and drug delivery. Electrochemotherapy (ECT) utilizes reversible electroporation to enhance transport of cell impermeant chemotherapy drugs, which has shown to be effective in treating brain tumors. For ECT treatments, reversible electroporation occurs at an electric field magnitude around 300 V/cm whereas IRE occurs above 500-600 V/cm.

Because ECT uses lower applied electric field magnitudes and fewer pulses than IRE, and the extent of electroporation largely depends on pulse duration and number, the zone of treatment is limited. IRE is more versatile, enabling control of the ablation zone in tissue. IRE is effective as a stand-alone therapy for ablating the primary tumor (e.g., can be administered with or without the need for chemotherapy drugs), yet still induces reversible electroporation further away from the electrodes, making cells susceptible to adjuvant therapies. Techniques have been explored for obtaining larger ablation volumes by capitalizing on the reversible electroporation zone achieved further away from the electrodes. A 2-3× larger zone of cell death using IRE treatment in combination with chemotherapeutic drugs, Bleomycin and Carboplatin, compared to IRE treatment alone was achieved in vitro. (Neal, R. E., Rossmeisl, J. H., D'Alfonso, V., Robertson, J. L., Garcia, P. A., Elankumaran, S., and Davalos, R. V., In vitro and numerical support for combinatorial irreversible electroporation and electrochemotherapy glioma treatment. Ann. Biomed. Eng. 42:475-487, 2014.)

Calcium in combination with electroporation has been explored by others to some extent. See, e.g., U.S. Application Publication No. 2019/0076528 and U.S. Pat. No. 9,943,599. It has been shown that ECT pulses used in conjunction with calcium cause more cell death and a greater decrease in cellular ATP than electroporation alone (Frandsen, S. K., H. Gissel, P. Hojman, T. Tramm, J. Eriksen, and J. Gehl. Direct therapeutic applications of calcium electroporation to effectively induce tumor necrosis. Cancer Res. 72:1336-41, 2012).

Previous work of the inventors (using 450 V, a frequency of 1 Hz, a pulse duration of 100 μs—with 80 pulses for IRE and 8 pulses for ECT) has shown that the lesion area for IRE or ECT treatment is greatly increased when CaCl₂ is added. (Wasson, E. M., Ivey, J. W., Verbridge, S. S., and Davalos, R. V., Ann. Biomed. Eng., 2017, 45, 2535-2547.)

Additionally, it has been previously shown that using nanosecond pulsed electric fields (nsPEFs) and a buffer containing sucrose and NaCl significantly decreased cell death when compared to a buffer containing calcium. (Pakhomova, O. N., Gregory, B., Semenov I., and Pakhomov, A. G., BBA—Biomembr., 2014, 1838, 2547-2554; see also, O. M. Nesin, O. N. Pakhomova, S. Xiao, A. G. Pakhomov, Manipulation of cell volume and membrane pore comparison following single cell permeabilization with 60- and 600-ns electric pulses, Biochim. Biophys. Acta Biomembr. 1808 (2011) 792-80.) More particularly, cells treated in a sucrose and NaCl buffer were rescued from cell swelling and early cell death while showing increased caspase activation. On the other hand, cells treated with CaCl₂, were initially spared from swelling, but were eventually overcome by CaCl₂ and experienced necrosis.

Even in light of previous work, the unpredictability in the field of treating tissue with electrical energy leaves questions unanswered. The invention described herein includes administering electrical energy treatments with lower voltages to avoid Joule heating and consequences relating thereto, while maintaining a sufficient level of efficacy. Additionally, the invention provides clinicians more options for administering selective electrical energy treatments and the ability to perform electrical energy treatments having a dual purpose (e.g., to kill cells in certain tissue regions while sparing cells in others and/or to irreversibly electroporate in certain regions while reversibly electroporating in others). With respect to electroporation applications involving immunotherapy in particular, this invention is beneficial to find additional ways of eliciting a positive immune response to enlist the patient's own body in further eliminating and/or preventing formation of tumors following electroporation treatment.

### SUMMARY OF THE INVENTION

To this end, the inventors have expanded on previous work relating to calcium IRE and have found ways of improving ablation efficacy and of improving patient safety/comfort. In particular, the inventors provide new protocols that are specific to accentuating the treatment margin without applying additional energy and which provide an advantage over microwave and radiofrequency ablation since the mechanism is non-thermal and spares vital structures.

Accordingly, the inventors provide electrical energy based methods for:

(i) selectively enhancing ablation in a tissue region, such as by increasing treatment margins, e.g., without increasing voltage;

(ii) selectively protecting a tissue region from effects of ablation, e.g., to reduce thermal damage, spare healthy or critical structures from damage, and/or allow for an enhanced reversible electroporation zone (such as for intracellular delivery of drugs across the blood-brain barrier) and/or enhanced electrochemotherapy;

(iii) selectively enhancing the IRE to thermal cell death ratio; and/or

(iv) delivering electrical pulses (such as IRE, H-FIRE, RE or ECT) in a manner to induce or elicit a positive/effective immune response.

With respect to H-FIRE in particular, instead of increasing voltage to obtain a larger area/volume of cell death, adjuvant calcium can be used to achieve comparable efficacy.

Alternatively, or in addition, a non-calcium containing buffer, such as sodium chloride and sucrose can offer protection for cells, allowing for selective ablation of tissue and/or for customization of treatment areas/zones and/or margin sizes and/or ablation geometry, e.g., surrounding the treatment zone. Accordingly, applications such as ECT can benefit from such protocols by providing for a more controlled area of reversible electroporation, with or without also administering irreversible electroporation in another region.

Applications involving reversible electroporation can also benefit from the presence of calcium during electroporation. The electric field magnitude during an IRE treatment decreases as the distance from the electrodes is increased. As such, typically a high electric field magnitude will develop close to the electrodes (irreversible electroporation, IRE) and a low electric field magnitude will develop far from the electrodes (reversible electroporation, RE). Cells in the IRE zone will die through loss of homeostasis resulting from IRE, but the inventors have found that the influx of calcium will cause cell death instead in the RE zone thereby increasing the area of the IRE zone. As such, the presence of excess or added calcium in combination with H-FIRE can provide not only for an enhanced IRE zone but also for an enhanced surrounding RE zone.

More particularly, embodiments of the invention include Aspect 1, which is a method of treating tissue comprising: applying a plurality of electrical pulses to a tissue region; and exposing the tissue region to one or more agent; wherein the applying of the electrical pulses is performed in a manner sufficient to treat cells of the tissue region with high-frequency irreversible electroporation (H-FIRE); and wherein the agent is capable of protecting cells from, enhancing or increasing, and/or inhibiting, decreasing, or limiting, one or more effects of the H-FIRE. For example, such methods can include methods of selectively treating cells that involve applying a plurality of electrical pulses to a tissue region; and exposing the tissue region to one or more agent comprising calcium; wherein the applying of the electrical pulses is performed in a manner sufficient to treat cells of the tissue region with high-frequency irreversible electroporation (H-FIRE); and wherein the agent is capable of protecting cells from, enhancing or increasing, and/or inhibiting, decreasing, or limiting, one or more effects of the H-FIRE, such as providing for an enhanced surrounding RE zone.

Aspect 2 is the method of Aspect 1, wherein one or more of the effects of the H-FIRE is chosen from cell death; quick cell death on the order of seconds or minutes; slow cell death on the order of hours, days, weeks, months or years; apoptosis; necrosis; heat; thermal effects; cell membrane permeabilization; inflammatory response; blood brain barrier disruption; transient blood brain barrier disruption; permanent damage; transient damage; immune response; a reversible electroporation zone and combinations thereof

Aspect 3 is the method of Aspect 1 or 2, wherein the agent comprises calcium (such as calcium chloride (CaCl₂), calcium acetate, calcium carbonate, calcium citrate, calcium phosphate, calcium propionate, calcium edetate, calcium malate, calcium bisglycinate, calcium gluconate, calcium glubionate, or any physiologically acceptable calcium salt capable of dissociating to contribute calcium ions) or a non-calcium containing buffer (such as a buffer comprising sucrose, e.g., sodium chloride (NaCl) plus sucrose). The calcium adjuvant or non-calcium containing adjuvant/buffer can be administered at an amount of about 50% of tissue/tumor volume, such as in the range of about 0.10% to 100% of tissue volume, or more, or from 0.5% to 99%, or from 1% to 95%, or from 20% to 90%, or 30% to 75%, or 25% to 60%, or 40% to 80% of the volume of the tissue region being treated, or any range in between any of these ranges or endpoints, including as endpoints any number encompassed thereby. In embodiments, the calcium adjuvant or non-calcium containing adjuvant/buffer can be administered at concentrations ranging from 0.1 mM to 500 mM, such as from 0.5 mM to 400 mM, or from 1 mM to 300 mM, or from 5 mM to 250 mM, or from 10 mM to 150 mM, or from 20 mM to 100 mM, such as from 2 mM to 15 mM, or 3 mM to 8 mM, or 5 mM to 7 mM, or 4 mM to 12 mM, or any range between any of these ranges or endpoints, including as endpoints any number encompassed thereby.

Aspect 4 is the method of any preceding Aspect, wherein the agent comprises calcium in an amount sufficient to: increase an area/zone and/or margin of ablation to a size comparable to that expected from H-FIRE administered using a higher voltage; and provide an increased IRE to thermal cell death ratio than without the calcium, such that a lower thermal effect and an enhanced positive immune response are provided.

Aspect 5 is the method of any preceding Aspect, wherein the positive immune response is promoted by immune cells present beyond the tissue region to which the plurality of electrical pulses are applied.

Aspect 6 is the method of any preceding Aspect, wherein one or more of the agents comprises calcium in an amount capable of increasing one or more effects of the H-FIRE in the tissue region, such as an excess amount of calcium.

Aspect 7 is the method of any preceding Aspect, wherein one or more of the agents is a protective agent and comprises a buffer capable of protecting cells from, inhibiting, decreasing, or limiting one or more effects of the H-FIRE, such as a buffer comprising sucrose, e.g., an NaCl buffer comprising sucrose.

Aspect 8 is the method of any preceding Aspect, wherein the exposing of the tissue region to the agent comprises: exposing a first tissue region to an agent comprising calcium in an amount sufficient to enhance one or more effects of the H-FIRE; and exposing a second tissue region to a non-calcium containing buffer in an amount sufficient to limit one or more effects of the H-FIRE.

Aspect 9 is the method of any preceding Aspect, wherein the first tissue region comprises cancer cells.

Aspect 10 is the method of any preceding Aspect, wherein the second tissue region comprises non-target cells, such as non-cancerous cells.

Aspect 11 is a method of treating tissue comprising: applying a plurality of electrical pulses to a tissue region, wherein the electrical pulses have a pulse width of less than 100 μs; and exposing the tissue region to one or more agent; wherein the applying is performed in a manner sufficient to cause electroporation of cells of at least a portion of the tissue region; and wherein the agent is capable of protecting cells from, enhancing or increasing, and/or inhibiting, decreasing, or limiting, one or more effects of the electroporation in at least a portion of the tissue region. In embodiments, the pulse widths can range from about 1 picosecond to 50 μs, such as about 10 ns to about 10 μs, or about 10 μs or less, and can be used in combination with the electrical pulses being administered at voltages ranging between 0 V to 10,000 V.

Aspect 12 is the method of Aspect 11, wherein the agent comprises calcium (such as calcium chloride (CaCl₂) or any physiologically acceptable calcium salt) or a non-calcium containing buffer (such as a buffer comprising sucrose, e.g., an NaCl buffer comprising sucrose).

Aspect 13 is the method of any preceding Aspect, wherein the plurality of electrical pulses are capable of electroporation based therapy, electroporation, irreversible electroporation (IRE), reversible electroporation (RE), electrochemotherapy (ECT), electrogenetherapy, supraporation, and/or high frequency irreversible (H-FIRE) electroporation, or combinations thereof.

Aspect 14 is the method of any preceding Aspect, wherein the applying and the exposing together provide for an increased IRE to thermal cell death ratio in at least a portion of the tissue region, as compared with the same treatment without the added agent, such that a lower thermal effect and an enhanced positive immune response are provided.

Aspect 15 is the method of any preceding Aspect, wherein the exposing of the tissue region to the agent comprises exposing the tissue region to an agent comprising calcium capable of enhancing one or more of the effects of the electroporation in at least a portion of the tissue region, as compared with the same treatment without the added agent.

Aspect 16 is the method of any preceding Aspect, wherein the exposing of the tissue region to the agent comprises exposing the tissue region to a non-calcium containing buffer capable of limiting one or more of the effects of the electroporation in at least a portion of the tissue region, as compared with the same treatment without the added agent.

Aspect 17 is a method of selectively treating cells, comprising: applying a plurality of electrical pulses to first and second tissue regions; exposing the first tissue region to a first agent in a manner such that more cell death occurs within the first tissue region than without presence of the first agent; and exposing the second tissue region to a second agent in a manner such that: less cell death, or no cell death, occurs within the second tissue region than without presence of the second agent; and/or the second tissue region comprises a zone of reversible electroporation, the zone being enhanced by presence of the second agent.

Aspect 18 is the method of Aspect 17, wherein the agent comprises calcium (such as calcium chloride, CaCl₂, or any physiologically acceptable calcium salt) or a non-calcium containing buffer (such as a buffer comprising sucrose, e.g., an NaCl buffer comprising sucrose), and can be administered at amounts/concentrations as outlined above according to Aspect 3.

Aspect 19 is the method of Aspect 17 or 18, wherein the applying and the exposing together provide for an increased IRE to thermal cell death ratio, such that a lower thermal effect and an enhanced positive immune response are provided.

Aspect 20 is the method of any preceding Aspect, wherein the tissue region (such as at least a portion of the first tissue region and/or the second tissue region) comprises: vasculature, nerve tissue, and/or tissue near the vasculature or the nerve tissue; and/or tissue near one or more electrodes used in applying the plurality of electrical pulses.

Aspect 21 is a system for selectively treating cells, comprising: at least one electrode; a voltage pulse generator coupled to the electrode; and one or more fluid delivery system capable of delivering a first agent to a first tissue region and a second agent to a second tissue region.

Aspect 22 is a method of selectively treating cells, comprising: applying a plurality of electrical pulses to first and/or second tissue regions; and exposing the first and/or second tissue region to one or more agent(s), which can be the same or different agents; wherein cells disposed in one of the first or second tissue regions are affected by the plurality of electrical pulses while cells disposed in the other tissue region are not so affected (e.g., cells disposed in one region are not affected in the same way and/or to the same extent as, and/or are affected differently than cells disposed in the other region).

Aspect 23 is the method of any of the preceding Aspects, wherein cells disposed in a first tissue region are affected by the plurality of electrical pulses and cells disposed in a second tissue region are not so affected.

Aspect 24 is the method of any of the preceding Aspects, wherein cells disposed in a second tissue region are affected by the plurality of electrical pulses and cells disposed in a first tissue region are not so affected.

Aspect 25 is the method of any of the preceding Aspects, wherein the plurality of electrical pulses comprises a delay or no delay between one or more successive pulses and/or between one or more successive bursts of pulses.

Aspect 26 is the method of any of the preceding Aspects, wherein the plurality of electrical pulses are capable of electroporation based therapy, electroporation, irreversible electroporation, reversible electroporation, electrochemotherapy, electrogenetherapy, supraporation, and/or high frequency irreversible electroporation, or combinations thereof, such as by way of a DC current.

Aspect 27 is the method of any of the preceding Aspects, wherein pulse length and/or delay (or no delay) between one or more pulses and/or bursts of the plurality of electrical pulses contribute(s) to the extent of a boundary between the first and second tissue regions (e.g., how large or small the first and/or second tissue region is in area or volume, and/or the relative size of each tissue region compared with the other).

Aspect 28 is the method of any of the preceding Aspects, wherein the agent is chosen from one or more adjuvant and/or buffer.

Aspect 29 is the method of any of the preceding Aspects, wherein the agent is a protective agent (e.g., the agent is administered in the second tissue region in an amount, and in a manner, and for a time sufficient to protect one or more cells disposed in the second tissue region from one or more effects of the plurality of electrical pulses).

Aspect 30 is the method of any of the preceding Aspects, wherein the agent is an enhancing agent (e.g., the agent is administered in the second tissue region in an amount, and in a manner, and for a time sufficient to enhance or increase one or more effect that the plurality of electrical pulses would have on one or more cells disposed in the second tissue region).

Aspect 31 is the method of any of the preceding Aspects, wherein the agent is an inhibiting agent (e.g., the agent is administered in the second tissue region in an amount, and in a manner, and for a time sufficient to inhibit, decrease, or limit one or more effect that the plurality of electrical pulses would have on one or more cells disposed in the second tissue region).

Aspect 32 is the method of any of the preceding Aspects, wherein one or more the effects of the plurality of electrical pulses is chosen from cell death, apoptosis, necrosis, slow cell death (on the order of hours, days or weeks or longer), quick cell death (on the order of seconds or minutes), heat, cell membrane permeabilization, inflammatory response, blood brain barrier disruption, transient blood brain barrier disruption, permanent damage, transient damage, a reversible electroporation zone and combinations thereof

Aspect 33 is the method of any of the preceding Aspects, wherein the agent is administered up to or more than 5 minutes, 15 minutes, 0.5 hr, 1 hr, 2 hr, 4 hr, 8 hr, or 12 hr before and/or after the applying of the plurality of electrical pulses, and/or the agent is administered during the applying of the plurality of electrical pulses.

Aspect 34 is the method of any of the preceding Aspects, wherein the applying is performed using a voltage for the plurality of electrical pulses of 0 V to 10,000 V, such as above 0 V or 1 V up to 10,000 V, and/or from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 5,000 V, up to 7,500 V, or for example from 100 V to 15,000 V, such as from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 15,000 V, up to 7,500 V, from 4,000 V to 12,000 V, such as less than 450 V, or less than 425 V, such as from above 0 V to 400 V, or from about 10 V to 350 V, or about 20 V to about 300 V, or about 30 V to about 250 V, or from about 15 V to about 200 V, or from about 50 V to about 150 V, or about 75 V to 100 V, or from 30 V to 225 V, or from 60 V to 375 V, or any range in between any of these ranges or endpoints, including as endpoints any number encompassed thereby.

Aspect 35 is the method of any of the preceding Aspects, wherein the applying is performed using at least two electrodes spaced from 0 cm to 10 cm apart, such as from above 0 cm up to 10 cm apart, or from 0.2 cm to 9 cm, such as from 0.5 cm to 5 cm, or from 1 cm to 4 cm apart, or from 2 cm to 3 cm, or 1.5 cm, or any range in between any of these ranges or endpoints, including as endpoints any number encompassed thereby.

Aspect 36 is the method of any preceding Aspect, wherein the electrodes are one or more needle, plate, surface or blunt tip electrodes or combinations thereof.

Aspect 37 is the method of any of the preceding Aspects, wherein the electrodes have a length (whether the length of the active tip of the electrode or the shaft of the probe) ranging from 1 cm to 30 cm, such as from 10 cm to 20 cm, or from 5 cm to 15 cm, and/or with a length of the active portion of the probe (e.g., energizable region) ranging from 0.5 cm to 10 cm, such as from 1 cm to 5 cm, or up to 3 cm or up to 4 cm, or any range in between any of these ranges or endpoints, including as endpoints any number encompassed thereby.

Aspect 38 is the method of any of the preceding Aspects, wherein one or more pulses of the plurality of electrical pulses have a pulse length in the picosecond to second range, such as in the nanosecond to millisecond range, or in the nanosecond to microsecond range, such as from 1 μs to 100 seconds, or from 1 μs to 100 ms, or from 1 ns to 100 μs, or below 100 μs, or below 10 μs, or from 1 μs to 1 μs, or below 1 μs, or from at least 0.1 μs up to 1 second, or from 0.5 μis up to 10 μs or up to 20 μs or up to 50 μs, such as 15, 25, 30, 35, 40, 55, 60, 75, 80, 90, 110, or 200 μs, or any range in between any of these ranges or endpoints, including as endpoints any number encompassed thereby, such as an H-FIRE burst scheme of pulse width and intra-phase delay ranging from 0.1 μs to 10 ms and an inter-pulse delay ranging from 0.1 μs to 1 s.

Aspect 39 is the method of any of the preceding Aspects, wherein the plurality of electrical pulses has a frequency in the range of 0 Hz to 100 MHz, such as from above 0 Hz or 1 Hz up to 100 MHz, such as from 2 Hz to 100 Hz, or from 3 Hz to 80 Hz, or from 4 Hz to 75 Hz, or from 15 Hz to 80 Hz, or from 20 Hz up to 60 Hz, or from 25 Hz to 33 Hz, or from 30 Hz to 55 Hz, or from 35 Hz to 40 Hz, or from 28 Hz to 52 Hz, or a frequency ranging from 100 Hz to 100 MHz, such as in the Hz range from 100 Hz or 1 Hz up to 100 Hz, or from 2 Hz to 100 Hz, or from 3 Hz to 80 Hz, or from 4 Hz to 75 Hz, or from 15 Hz to 80 Hz, or from 20 Hz to 60 Hz, or from 25 Hz to 33 Hz, or from 30 Hz to 55 Hz, or from 35 Hz to 40 Hz, or from 28 Hz to 52 Hz, or a frequency in the kHz or MHz range, such as from 1 kHz to 10 kHz, or from 2 kHz to 8 kHz, or from 3 kHz to 5 kHz, or from 4 kHz to 15 kHz, or from 6 kHz to 20 kHz, or from 12 kHz to 30 kHz, or from 25 kHz to 40 kHz, or from 5 kHz to 55 kHz, or from 50 kHz to 2 MHz, including any range in between, such as from 75 kHz to 150 kHz, or from 100 kHz to 175 kHz, or from 200 kHz to 250 kHz, or from 225 kHz to 500 kHz, or from 250 kHz to 750 kHz, or from 500 kHz to 1 MHz, or any range in between any of these ranges or endpoints, including as endpoints any number encompassed thereby.

Aspect 40 is the method of any of the preceding Aspects, wherein the plurality of electrical pulses have a waveform that is square, triangular, trapezoidal, exponential decay, sawtooth, sinusoidal, and/or alternating polarity.

Aspect 41 is the method of any of the preceding Aspects, wherein the plurality of electrical pulses have a total number of pulses, and/or a total number of pulses per burst, ranging from 1-5,000 pulses, such as from at least 1 up to 3,000 pulses, or at least 2 up to 2,000 pulses, or at least 5 up to 1,000 pulses, or at least 10 up to 500 pulses, or from 10 to 100 pulses, such as from 20 to 75 pulses, or from 30 to 50 pulses, such as 1, 5, 10, 15, 20, 25, 30, 35, 40, 45, 50, 60, 70, or 90 pulses, or the total number of pulses and/or bursts can range from 1 to 5,000 pulses/bursts, such as from at least 1 up to 3,000 pulses/bursts, or at least 2 up to 2,000 pulses/bursts, or at least 5 up to 1,000 pulses/bursts, or at least 10 up to 500 pulses/bursts, or from 10 to 100 pulses/bursts, such as from 20 to 75 pulses/bursts, or from 30 to 50 pulses/bursts, such as 1, 5, 10, 15, 20, 25, 30, 35, 40, 45, 50, 60, 70, or 90 pulses/bursts, or any range in between any of these ranges or endpoints, including as endpoints any number encompassed thereby.

Aspect 42 is the method of any preceding Aspect, wherein there is a delay between pulses, and/or a delay between bursts, and/or a delay within a pulse/burst, and/or a delay between the polarity switch within a pulse/burst and the delay can be on the order of microseconds or seconds, such as one to one thousand microseconds, such as 1, 2, 3, 4, 5, 6, 7, 8, 9, 10, 15, 20, 25, 30, 35, 40, 45, 50, 75, 100, 150, 200, 250, 300, 350, 400, 450, 500, 600, 700, 800, 900, or 1,000 microseconds or one to several seconds such as 1, 1.5, 2, 2.5, 3. 3.5, 4, 4.5, 5, 5.5, 6, 6.5, 7, 7.5, 8, 8.5, 9, 9.5, 10, 11, 12, 13, 14, 15, 16, 17, 18, 19, 20, 22, 24, 26, 28, 30 seconds or more. Cumulatively, the one or more delays may be on the order of seconds or minutes.

Aspect 43 is the method of any of the preceding Aspects, wherein the plurality of electrical pulses comprises one or more unipolar and/or one or more bipolar pulses and/or one or more pulse with a positive polarity and/or one or more pulse with a negative polarity, and/or one or more pulse with alternating polarity.

Aspect 44 is the method of any of the preceding Aspects, wherein the plurality of electrical pulses is administered from one or more electrode, whether in combination or not with a surface electrode and/or ground electrode.

Aspect 45 is the method of any of the preceding Aspects, wherein the plurality of electrical pulses is administered from two or more electrodes (e.g., two or more electrodes disposed in contact with one or the other tissue region, or two or more electrodes disposed in each or both), and from any number of electrodes, such as 1, 2, 3, 4, 5, 6, 7, 8, 9, 10, 11, 12, 13, 14, 15, 16, 17, 18, 19, or 20 electrodes and in any configuration relative to one another.

Aspect 46 is the method of any of the preceding Aspects, wherein exposure of one or more of the cells to one or more of the adjuvants/agents results in cell death.

Aspect 47 is the method of any of the preceding Aspects, wherein exposure of one or more of the cells to one or more of the buffers results in reduced cell death.

Aspect 48 is the method of any of the preceding Aspects, wherein the adjuvant comprises calcium, such as calcium chloride (CaCl₂), or in the form of any calcium salt, especially physiologically acceptable/compatible calcium salts.

Aspect 49 is the method of any preceding Aspect, wherein the buffer comprises a non calcium containing buffer, such as a buffer comprising sucrose (e.g., NaCl and sucrose).

Aspect 50 is the method of any of the preceding Aspects, wherein one or more of the electrodes provide for injection of CaCl₂ solution.

Aspect 51 is the method of any of the preceding Aspects, wherein imaging is used to guide or confirm desired placement of one or more electrode and/or administration of one or more agent, such as a calcium containing agent and/or one or more non-calcium containing agent (such as a buffer comprising sucrose, e.g., an NaCl buffer comprising sucrose) into one or more of the first and/or second tissue regions.

Aspect 52 is the method of any of the preceding Aspects, wherein one or more agent, buffer, and/or adjuvant is introduced to one or more tissue region by administration in any manner, such as by injection, infusion, or exposure, such as parenteral, intravenous, intraarterial, intradermal, transdermal, intranasal, local or intralesional, intraperitoneal, intramuscular, buccal, oral, or transmucosal administration. In embodiments, direct tumor injection of the calcium adjuvant and/or non-calcium containing adjuvant/buffer can be performed, for example, by disposing a needle for injecting the solution at various positions within the tissue/tumor to allow for uniform and/or selective distribution of the agent. Co-delivery of the adjuvant and/or buffer with electroporation treatment, for example using a needle/electrode system, is also possible.

Aspect 53 is the method of any of the preceding Aspects, wherein one or more cells treated with adjuvant are cancer cells.

Aspect 54 is the method of any of the preceding Aspects, wherein one or more cells treated with an adjuvant or agent that is a non-calcium buffer, such as a buffer comprising sucrose, e.g., an NaCl buffer comprising sucrose, are non-cancerous cells and/or non-target cells.

Aspect 55 is the method of any of the preceding Aspects, wherein one or more tissue region is brain, prostate, ovarian, cervical, liver, kidney, pancreatic, gall bladder, stomach, heart, esophageal, intestinal, lung, dermal, epithelial, connective, or muscle tissue.

Aspect 56 is the method of any preceding claim, wherein one or more tissue region comprises endothelial cells.

Aspect 57 is the method of any of the preceding Aspects, wherein one or more calcium-containing adjuvant is delivered to a tumor and/or one or more non-calcium containing buffer is delivered to vasculature.

Aspect 58 is a system for selectively treating cells, comprising: at least one electrode; a voltage pulse generator coupled to the electrode and configured to perform one or more method of any of the preceding Aspects in whole or part, such as by comprising programming to accomplish any one or more elements or steps of any one or more of the preceding Aspects.

### BREIF DESCRIPTION OF THE DRAWINGS

The accompanying drawings illustrate certain aspects of embodiments of the present invention, and should not be used to limit the invention. Together with the written description, the drawings serve to explain certain principles of the invention.

FIG. 1A is a diagram depicting a generic unipolar pulse scheme for electrical energy based therapies, such as IRE.

FIG. 1B is a diagram depicting representative pulse schemes for H-FIRE comprising a desired number of bursts of bipolar pulses delivered with a desired applied voltage, a desired pulse width (e.g., 1, 2, 5, or 10 μs), a desired waveform (e.g., square), a desired inter-pulse delay (e.g., 1 μs), a pulse number adjusted for a total on time per burst (e.g., 100 μs) (respectively, 100, 50, 20, or 10 pulses), and a desired frequency (such as a frequency of one burst per second).

FIG. 2 is a schematic depicting custom made electrodes inserted into a collagen scaffold platform prior to pulsing with electrode spacing of 4.0 mm (scale bar 4 mm).

FIGS. 3A-B are illustrations depicting the electric field distribution (FIG. 3A) and the corresponding temperature distribution (FIG. 3B) in a collagen scaffold.

FIG. 4 is a schematic showing the sizes of lesions resulting from various electroporation treatments with calcium chloride and sodium chloride.

FIG. 5 is a graph showing the calculated lesion area for collagen scaffold experiments demonstrating that calcium chloride treatments lead to larger lesions than NaCl controls.

FIG. 6 is a graph showing the calculated electric field thresholds for collagen scaffold experiments demonstrating that calcium chloride treatments lead to lower electric field thresholds resulting in cell death than their sodium chloride controls.

FIG. 7 is a schematic showing the reversible electroporation zones for cells stained immediately following electroporation treatment.

FIG. 8 is a graph showing the calculated reversible electroporation zone for collagen scaffold experiments demonstrating that calcium chloride treatments result in larger reversible electroporation zones.

FIG. 9 is a schematic overlaying the reversible electroporation zone (white circle) with the irreversible electroporation zone (darker zone within the white circle) to show the calcium treatments result in smaller reversible:irreversible electroporation zone ratios. The region beyond the white circled area is non-electroporated tissue.

FIG. 10 is a graph showing the ratio of reversible electroporation to irreversible ablation (IRE) areas for collagen scaffold experiments demonstrating that calcium chloride treatments result in smaller reversible:irreversible electroporation zone ratios.

FIG. 11 is a schematic showing cell death following H-FIRE treatment for cells treated with solutions of sodium chloride, potassium chloride, calcium chloride, and a mixture of calcium chloride and sodium chloride.

FIG. 12 is a graph showing the ablation areas for cells treated with sodium chloride, potassium chloride, calcium chloride, and a mixture of calcium chloride and sodium chloride during H-FIRE treatment.

FIG. 13 is an illustration comparing the areas of thermally damaged cells, electroporated cells, and surviving cells following H-FIRE treatment at various voltages with and without calcium chloride.

FIGS. 14-18 are flowcharts illustrating various exemplary methods of the invention.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather the following discussion is provided to give the reader a more detailed understanding of certain aspects and features of the invention.

Throughout the present teachings, any and all of the features and/or components disclosed or suggested herein, explicitly or implicitly, may be practiced and/or implemented in any combination, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the inventions presented herein or in any future applications claiming priority to the instant application.

Embodiments of the present invention provide electrical energy based treatment protocols (e.g., H-FIRE, IRE, RE and/or ECT) with the administration of one or more agent(s) to enhance the zone of electroporation and/or to protect a region from electroporation and/or one or more effects of electroporation.

In embodiments, the agent administered is adjuvant calcium. In another embodiment, the agent administered is a non-calcium buffer. In another embodiment, both adjuvant calcium and non-calcium buffer are delivered to the tissue region and/or cells receiving electroporation treatment. In another embodiment, the treatment is delivered in a manner promoting an immune response, such as an enhanced positive immune response, by way of immune cells that respond to tissue injury resulting from electroporation.

Definitions:

The term “pulse” refers to an electrical signal with a single phase (monopolar, unipolar) or more than one phase (bi-polar). If bi-polar, there can be a delay between phases or the switch between phases/polarity can be immediate (no intra-pulse delay).

The term “burst” refers to a set of pulses, a group of pulses, or a pulse group.

The term “inter-pulse delay” refers to the condition where no energy is applied for a period of time between one pulse or set of pulses and another pulse, or between one bi-polar pulse or set of bi-polar pulses and another pulse or bi-polar pulse.

The term “total on time” refers to the time associated with energizing an electrode. For example, for a 5-1-5 waveform comprising a bipolar pulse with a 5 μs pulse width and a 1 μs inter-pulse delay, a burst of 20 pulses would have a total on time of 100 μs.

The term “thermal damage” refers to damage to a treated tissue caused by an increase in temperature which results in death of the tissue and/or denaturing of proteins.

The terms “selective” ablation, electroporation, administration or treatment; “selectively” ablating, electroporating, treating or administering electrical energy; “controlled” electroporation, ablation, treatment, or administering of electrical energy; treatment protocols with a “dual purpose” and the like are used to refer to performing an electrical energy treatment protocol in a manner designed to obtain a desired effect in one tissue area while also obtaining a desired effect in another tissue area. The treatment can have the same type of effect (e.g., H-FIRE) in both tissue regions but with a different level of efficacy (e.g., different size treatment zones), or the treatment can have a different type of effect in each tissue region (e.g., IRE in one region and RE in another region) with a different purpose (e.g., kill cells in one region and spare cells in another, or kill cells in one region and allow for uptake of agents by RE mechanisms in another region).

The term “agent” refers to any substance, composition, or solution administered as an adjuvant in combination with administering electrical energy treatment. Agents include calcium adjuvants for enhancing or increasing one or more effects of the electrical energy treatment and buffers, such as non-calcium buffers (such as a buffer comprising sucrose, e.g., an NaCl buffer comprising sucrose) for protecting cells from and/or inhibiting, decreasing, or limiting one or more effects of the electrical energy treatment. The terms “agent,” “adjuvant,” “buffer,” “substance,” “composition,” or “solution” may be used interchangeably in the context of this disclosure.

In the following examples, the inventors demonstrate that it is possible to increase ablation size and/or treatment areas/zones and/or margins using electroporation with adjuvant calcium, and that using an adjuvant such as a non-calcium containing buffer, such as a sodium chloride and sucrose buffer, offers protection for cells, allowing for selective ablation or treatment, thereby enhancing the safety and efficacy of treatment.

As the results show for example, using a 10 μs H-FIRE pulse with calcium lowers the electric field threshold to a value comparable to an IRE treatment. This would allow the use of clinically available generator to be used and negate the need for custom electronics, making treatment more accessible. Additionally, calcium chloride is non-toxic to cells at the concentrations used, unlike chemotherapeutics that have been used previously in ECT treatments and IRE treatments to increase ablation sizes. Finally, if tumors are located in complex organs such as the pancreas or brain that contain many blood vessels/nerves/neurons, then ablation can be contained/controlled and/or selectively administered to preserve critical structures.

Scaffold Preparation

U251 malignant glioma cells (Sigma Aldrich, 09063001) were maintained at 5% CO₂ and 37° C. in Eagle's Minimum Essential Medium (Sigma Aldrich) supplemented with 1% penicillin/streptomycin (Life Technologies), 10% fetal bovine serum (Atlanta Biologicals), 1% non-essential amino acids (Sigma Aldrich) and 1 mM sodium pyruvate (Sigma Aldrich). Cells were routinely passaged at 80-90% confluence.

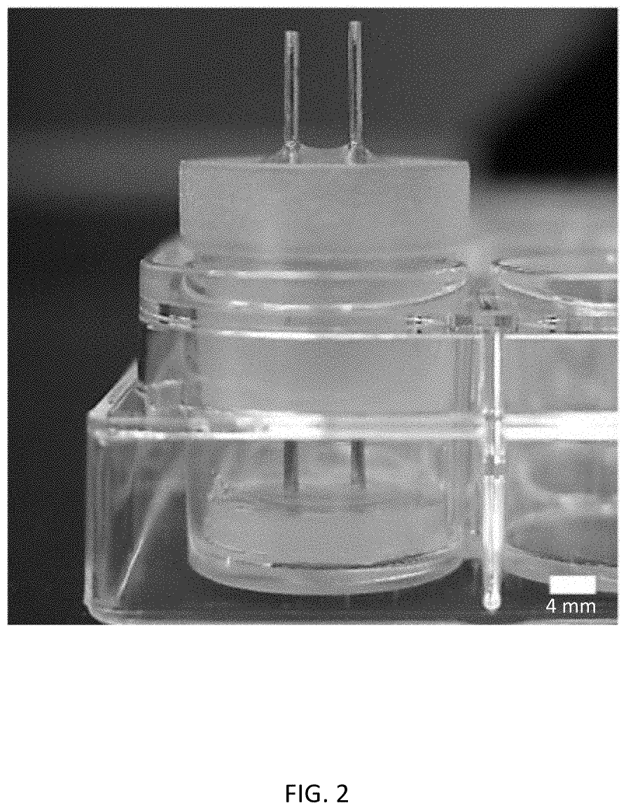

Sterile polydimethylsiloxane (PDMS, SYLGARDTM 184, Dow Corning) wells (10 mm diameter, 1 mm height) were placed in a 24 well plate to ensure uniform collagen scaffold geometry and electric field distribution between each replicate. PDMS wells were treated with 1% PEI (Acros Organics) for 10 min, 0.1% glutaraldehyde (Fisher Scientific) for 20 min, and then washed twice with deionized water prior to collagen seeding to ensure collagen adhesion during treatment. Commercial rat tail collagen type I (BD Biosciences) was neutralized using a solution of 10× Dulbecco's Modified Eagle Medium (10% total volume, Sigma Aldrich), 1 N NaOH (2% collagen volume, Sigma Aldrich), and 1× Dulbecco's Modified Eagle Medium (Sigma Aldrich) to a final concentration of 5 mg/mL. U251 cells were detached from flasks using 0.25% trypsin/EDTA (Thermo Fisher Scientific) solution and added to the neutralized collagen solution at a concentration of 1×10⁶ cells/mL. The collagen/cell solution was dispensed into PDMS wells and PDMS tops were used to mold the collagen flat while they polymerized in a cell culture incubator for 20 min. PDMS tops were then removed and cell culture media was added. Collagen scaffolds were maintained in the incubator for 24 hr prior to treatment. For further reference, FIG. 2 shows a representative experimental platform.

With respect to H-FIRE in particular, embodiments of the invention include a method of treating tissue comprising: applying a plurality of electrical pulses to a tissue region; and exposing the tissue region to one or more agent; wherein the applying of the electrical pulses is performed in a manner sufficient to treat cells of the tissue region with high-frequency irreversible electroporation (H-FIRE); and wherein the agent is capable of protecting cells from, enhancing or increasing, and/or inhibiting, decreasing, or limiting, one or more effects of the H-FIRE. The agent can comprise calcium and/or a non-calcium containing buffer with sucrose.

Adjuvant and Electroporation Treatment

In this example, after 24 h, cell culture media was aspirated and replaced with either CaCl₂, NaCl, KCl, or a combination of CaCl₂ and NaCl solutions (1 mM or 5 mM) and allowed to incubate for 30 minutes to saturate the collagen scaffold. All solutions consisted of the same base ingredients: 250 mM sucrose, 1 mM MgCl₂, and 10 mM HEPES buffer in deionized water with a pH in the range of 7.2-7.4. These solutions were then removed and fresh CaCl₂, NaCl, KCl, or CaCl₂/NaCl solutions were allowed to incubate for another 10 minutes to ensure all cell culture media was washed out of the scaffold. Cell culture media contains things such as serum and antibiotics that may affect results. Finally, fresh solutions were added immediately prior to pulsing. All solutions were adjusted to have a pH between 7.2-7.4. The osmolarity and conductivity of the buffers used are shown in Table 1:

TABLE 1
Properties of solutions used in this invention

| | | Conductivity | Osmolarity |
|---|---|---|---|
| Concentration | Solution | (S/m) | (mOsm/L) |
| 1 mM | CaCl₂ | 0.075 ± 0.004 | 289 |
| | NaCl | 0.056 ± 0.001 | 287 |
| | KCl | 0.064 | 289 |
| | CaCl₂ + NaCl | 0.065 | 287 |
| 5 mM | CaCl₂ | 0.131 | 300 |
| | NaCl | 0.089 | 291 |

In embodiments, one or more of the agents comprises calcium in an amount (such as from 0.1 mM to 500 mM) capable of enhancing and/or increasing one or more effects of the H-FIRE in the tissue region, and/or one or more of the agents comprises sucrose, or a combination of NaCl and sucrose, in an amount (such as from 0.1 mM to 500 mM) capable of protecting cells from, inhibiting, decreasing, or limiting one or more effects of the H-FIRE.

In practice, in vivo, the adjuvant calcium and/or non-calcium containing buffer can be administered in any manner, such as by injection, infusion, or exposure, such as parenteral, intravenous, intraarterial, intradermal, transdermal, intranasal, local or intralesional, intraperitoneal, intramuscular, buccal, oral, or transmucosal administration, depending on the particular tissue or application. Adjuvant calcium and/or non-calcium containing buffer can be administered immediately prior, such as within 5 minutes, 1 minute, less than 30 seconds prior, or up to or more than 5 minutes, 15 minutes, 0.5 hr, 1 hr, 2 hr, 4 hr, 8 hr, or 12 hr before and/or after the applying of the plurality of electrical pulses, and/or the agent can be administered during the applying of the plurality of electrical pulses. In embodiments, the most effective course of action would be to inject the adjuvant directly into the tissue immediately before treatment to limit the amount of diffusion out of the treatment area before the electrical energy treatment begins. Injecting after pulsing may contribute to a loss of some efficacy as reversibly electroporated cells recover within minutes of removing electric field.

The calcium adjuvant or non-calcium containing adjuvant/buffer can be administered at an amount of about 50% of tissue/tumor volume, such as in the range of about 0.10% to 100% of tissue volume, or more, or from 0.5% to 99%, or from 1% to 95%, or from 20% to 90%, or 30% to 75%, or 25% to 60%, or 40% to 80% of the volume of the tissue region being treated, or any range in between any of these ranges or endpoints, including as endpoints any number encompassed thereby. The calcium adjuvant or non-calcium containing adjuvant/buffer can be administered at concentrations ranging from 0.1 mM to 500 mM, such as from 0.5 mM to 400 mM, or from 1 mM to 300 mM, or from 5 mM to 250 mM, or from 10 mM to 150 mM, or from 20 mM to 100 mM, such as from 2 mM to 15 mM, or 3 mM to 8 mM, or 5 mM to 7 mM, or 4 mM to 12 mM, or any range between any of these ranges or endpoints, including as endpoints any number encompassed thereby.

In this example, two hollow, stainless-steel needle electrodes (Howard electronics) were inserted into the scaffolds using a custom designed electrode housing. The electrodes had an outer diameter of 0.914 mm and inner diameter of 0.635 mm and were spaced 4 mm apart (center-to-center) (FIG. 2). H-FIRE pulses were delivered using a high-voltage pulse generator (EPULSUS®-FBM1-5, EnergyPulse Systems, Lda). Voltage waveforms were captured using a high voltage probe and oscilloscope (DPO 2012, Tektronix). In embodiments, the plurality of electrical pulses is administered from two or more electrodes (e.g., two or more electrodes disposed in contact with one or another tissue region (such as a needle electrode disposed within a target region and a surface electrode disposed in contact with skin), or two or more electrodes disposed in each or both regions to be treated), and from any number of electrodes, such as 1, 2, 3, 4, 5, 6, 7, 8, 9, 10, 11, 12, 13, 14, 15, 16, 17, 18, 19, or 20 electrodes and in any configuration relative to one another. For example, the electrodes can be spaced apart a distance of from 0 cm to 10 cm apart, such as from above 0 cm up to 10 cm apart, or from 0.2 cm to 9 cm, such as from 0.5 cm to 5 cm, or from 1 cm to 4 cm apart, or from 2 cm to 3 cm, or 1.5 cm, or any range in between any of these ranges or endpoints, including as endpoints any number encompassed thereby.

In any embodiment administering electrical energy described herein, the electrodes can be one or more needle electrodes, plate electrodes, surface electrodes, hollow electrodes, blunt tip electrodes or combinations thereof. For example, in embodiments, the adjuvant(s) can be administered through hollow electrodes and/or electrodes/probes with hollow channels configured for the administration of one or more fluids, such as disclosed in U.S. Pat. No. 10,245,098. In embodiments, the electrodes can have a length (whether the length of the active tip of the electrode or the shaft of the probe) ranging from 1 cm to 30 cm, such as from 10 cm to 20 cm, or from 5 cm to 15 cm, and/or with a length of the active portion of the probe (e.g., energizable region) ranging from 0.5 cm to 10 cm, such as from 1 cm to 5 cm, or from 2 cm to 6 cm, or 1.5 cm to 8 cm, or up to 3 cm or up to 4 cm, or any range in between any of these ranges or endpoints, including as endpoints any number encompassed thereby.

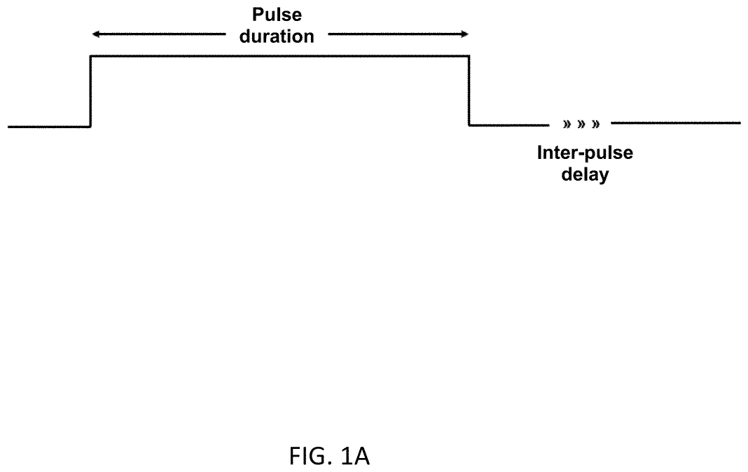

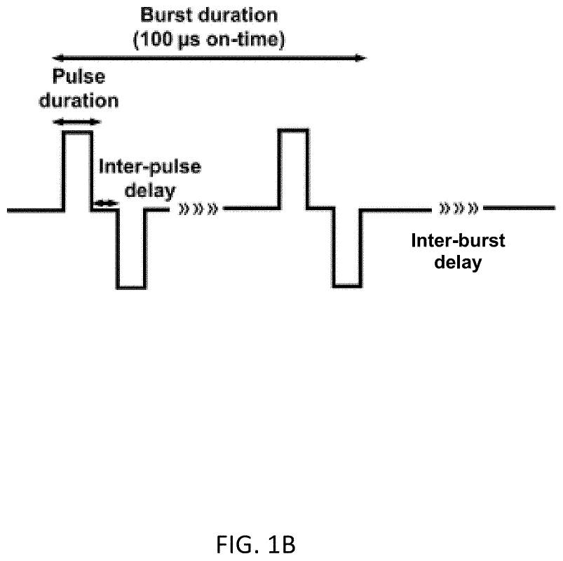

The pulses delivered were bipolar pulses having a positive pulse duration, inter-pulse delay, and negative pulse duration (see, e.g., FIG. 1B). Inter-pulse delay was kept constant (1 μs) while pulse width was varied (1, 2, 5, 10 μs). A total of eighty (80) bursts were delivered at 800 V for a total on time of 100 μs per burst (respectively, a total number of 100, 50, 20, 10 pulses per burst) at a frequency of one burst per second.

Depending on the particular application and other protocol parameters, such as electrode spacing, any voltage can be applied, including for example in the range of 0 V to 10,000 V, such as above 0 V or 1 V up to 10,000 V, and/or from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 5,000 V, up to 7,500 V, or for example from 100 V to 15,000 V, such as from 500 V up to 3,000 V, and/or from 1,000 V up to 2,000 V, such as up to 250 V, up to 300 V, up to 350 V, up to 600 V, up to 650 V, up to 800 V, up to 1,200 V, up to 1,500 V, up to 15,000 V, up to 7,500 V, from 4,000 V to 12,000 V, such as less than 450 V, or less than 425 V, such as from above 0 V to 400 V, or from about 10 V to 350 V, or about 20 V to about 300 V, or about 30 V to about 250 V, or from about 15 V to about 200 V, or from about 50 V to about 150 V, or about 75 V to 100 V, or from 30 V to 225 V, or from 60 V to 375 V. Additionally, or alternatively, for example, the pulse widths can range from about 1 picosecond to 50 microseconds, such as about 10 ns to about 10 microseconds, or about 10 microseconds or less.

or any range in between any of these ranges or endpoints, including as endpoints any number encompassed thereby.

With respect to H-FIRE in particular, one or more pulses of the plurality of electrical pulses can have a pulse length in the picosecond to microsecond range, such as in the nanosecond to microsecond range, including from 1 picosecond to below 10 microseconds, or from 1 picosecond to 1 microsecond, or below 1 microsecond, or from at least 0.1 microsecond up to 5 microseconds, or from 0.5 microseconds up to 2 microseconds or up to 10 microseconds, or any range in between any of these ranges or endpoints, including as endpoints any number encompassed thereby, such as a high-frequency irreversible electroporation burst scheme of pulse width and intra-phase delay ranging from 0.1 μs to 10 ms and an inter-pulse delay ranging from 0.1 μs to 1 s. The pulses can be unipolar or bi-polar. Any desired waveform can also be used, including square, triangular, trapezoidal, exponential decay, sawtooth, sinusoidal, and/or such waveforms comprising one or more pulses of alternating polarity.

Additionally, the pulsing schemes can incorporate one or more intra- or inter-pulse delays and/or one or more intra- or inter-burst delays. For example, pulsing schemes of bursts of pulses comprising schemes of 1-1-1 μs, 2-1-2 μs, 5-1-5 μs, or 10-1-10 μs with up to a 1-second delay between bursts can be used. In general, for H-FIRE, pulsing schemes conforming to the following formula can be used: (i) administering a pulse with a first polarity and a pulse duration of less than 10 microseconds, (ii) administering a delay with a duration of up to 20 microseconds, (iii) administering a pulse with a second polarity (that can be the same or a different polarity than the first pulse) and a pulse duration of less than 10 microseconds, (iv) administering a delay of up to 1 second, then (v) repeating the administering of (i)-(iv) a desired number of times.

Any number of pulses can be administered wherein there are a total number of pulses, and/or a total number of pulses per burst, ranging from 1-5,000 pulses, such as from at least 1 up to 3,000 pulses, or at least 2 up to 2,000 pulses, or at least 5 up to 1,000 pulses, or at least 10 up to 500 pulses, or from 10 to 100 pulses, such as from 20 to 75 pulses, or from 30 to 50 pulses, such as 1, 5, 10, 15, 20, 25, 30, 35, 40, 45, 50, 60, 70, or 90 pulses, or the total number of pulses and/or bursts can range from 1 to 5,000 pulses/bursts, such as from at least 1 up to 3,000 pulses/bursts, or at least 2 up to 2,000 pulses/bursts, or at least 5 up to 1,000 pulses/bursts, or at least 10 up to 500 pulses/bursts, or from 10 to 100 pulses/bursts, such as from 20 to 75 pulses/bursts, or from 30 to 50 pulses/bursts, such as 1, 5, 10, 15, 20, 25, 30, 35, 40, 45, 50, 60, 70, or 90 pulses/bursts, or any range in between any of these ranges or endpoints, including as endpoints any number encompassed thereby.

In embodiments, one or more of the effects of the H-FIRE that is enhanced, limited or prevented is chosen from cell death; quick cell death on the order of seconds or minutes; slow cell death on the order of hours, days, weeks, months or years; apoptosis; necrosis; heat; thermal effects; cell membrane permeabilization; inflammatory response; blood brain barrier disruption; transient blood brain barrier disruption; permanent damage; transient damage; immune response; a reversible electroporation zone and combinations thereof.

Of particular interest are such methods, wherein the agent comprises calcium in an amount sufficient to: increase an area or margin of ablation to a size comparable to that expected from H-FIRE administered using a higher voltage; and/or provide an increased IRE to thermal cell death ratio than without the calcium, such that a lower thermal effect and an enhanced positive immune response are provided. Additionally, or alternatively, such methods can include promoting a positive immune response, such as by immune cells present beyond the tissue region to which the plurality of electrical pulses are applied. For example, in embodiments, a more positive immune response can be promoted by administering calcium in combination with any one or more of the electroporation methods and protocols, or portions thereof, disclosed in U.S. Patent Application Publication No. 2019/0282294, which is incorporated by reference herein in its entirety. Further, it has been shown that H-FIRE effectively ablates the primary tumor and induces a pro-inflammatory shift in the tumor microenvironment; that local treatment with H-FIRE significantly reduces 4T1 metastases; that H-FIRE kills 4T1 cells through non-thermal mechanisms associated with necrosis and pyroptosis resulting in damage associated molecular pattern signaling in vitro and in vivo; that that the level of tumor ablation correlates with increased activation of cellular immunity; that the decrease in metastatic lesions is dependent on the intact immune system and H-FIRE generates 4T1 neoantigens that engage the adaptive immune system to significantly attenuate tumor progression; and that the non-thermal cell death mechanism of H-FIRE has been shown to elicit a more positive immune response after treatment when compared to modalities that rely on heating such as radiofrequency ablation, microwave ablation, or high-intensity focused ultrasound. See, e.g., Ringel-Scaia, V. et al., “High-frequency irreversible electroporation is an effective tumor ablation strategy that induces immunologic cell death and promotes systemic anti-tumor immunity,” EBioMedicine, 2019 June; 44: 112-125, which is incorporated by reference herein in its entirety. Thus, combining calcium administration with any one or more of these methods/protocols, or portions thereof, can be used to promote a more positive or enhanced immune response.

After pulsing, CaCl₂, NaCl, KCl, and CaCl₂/NaCl solutions were removed and cell culture media was added to each scaffold. For irreversible threshold experiments, the scaffolds were returned to the incubator for 24 hr prior to live/dead staining. For reversible threshold experiments, scaffolds were imaged immediately after treatment.

Analysis of Treatment Areas and Margins, Ablation Size and Efficacy

To visualize ablations, a live/dead stain was performed to visualize live and dead regions of cells in the scaffold. Scaffolds were incubated with 2 μM Calcein AM and 23 μM propidium iodide in phosphate buffered saline (PBS) for 30 minutes at room temperature. Scaffolds were then washed 2× with PBS prior to imaging using an inverted microscope (DMI 6000B, Leica Microsystems) with a 5× objective. Lesion areas were quantified for each treatment using a MATLAB algorithm. Since 24 h post treatment is sufficient to allow any reversibly electroporated cells to recover (as pore resealing happens on the order of minutes), staining the cells immediately after treatment and 24 h later allows for quantification of both the reversible zone of electroporation and irreversible zones of ablation.

To stain the scaffolds, media was removed and replaced with phosphate buffered saline (PBS) containing 2 μM Calcein AM (Invitrogen) and 23 μM PI (Invitrogen) and incubated at room temperature for 30 min. Scaffolds were then washed twice with PBS prior to imaging using an inverted microscope (DMI 6000B, Leica Microsystems) with a 5× objective, filter cubes, and an EM-CCD camera (Hamamatsu C9100). The appropriate filters were used to image Calcein AM (Ex:460-500, DC: 505, EM: 570-640) and propidium iodide (EX:545/26, DC:565, EM:605/70). To determine the reversible zone of electroporation, treatment followed the same electroporation protocol, but Calcein AM and PI were added to the CaCl₂ and NaCl solutions at all steps prior to pulsing. Scaffolds were then imaged immediately after treatment.

For analysis, images were separated into two channels (green—Calcein AM, red—PI) and ablation areas for the green channel were analyzed for irreversible thresholds and red channels for reversible electroporation thresholds using a custom algorithm written in MATLAB. In cases where the algorithm was unable to accurately measure ablations, coordinate points from the algorithm outlining the ablation area were used as a guide to manually trace and measure the area in ImageJ. Ablation area measurements were mapped to a finite element model of the experimental setup to determine the corresponding electric field threshold.

Irreversible electroporation experiments were repeated 6-12 times for each condition. Reversible electroporation experiments were repeated at least six times. Discrepancies in the number of replicates between conditions were due to bubbles or other defects that may have changed the electric field distribution or prohibited a reliable measurement of ablation area. These scaffolds were excluded from analysis. Two-way ANOVA was used to test for differences in cell death area due to the different applied solutions and pulse waveforms. Tukey post-hoc comparisons were used to examine differences among treatment groups. Statistical analyses were performed with a confidence level of α=0.05 (MP Pro 14). Results are shown as means±standard deviation.

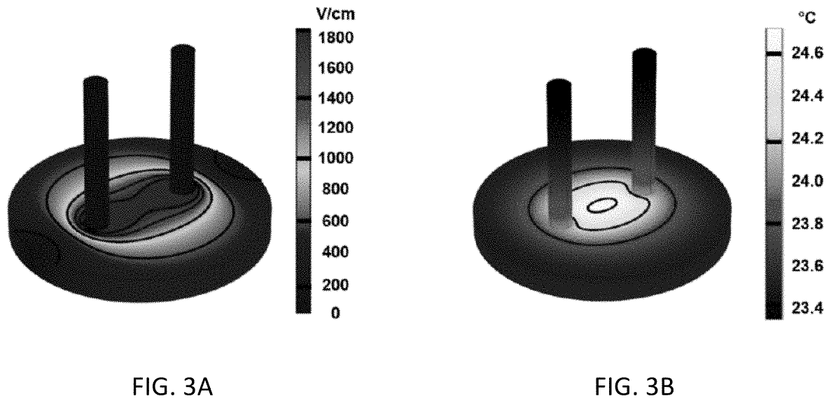

Treatments were simulated using the finite element method (COMSOL Multiphysics, Burlington, Mass.) and the electric field and temperature distributions were determined. The mesh was refined until there was <1% change in the electric field and temperature values along a cutline between the electrodes. The mesh consisted of 102,615 total elements. It was determined that temperature only increased 4.4° C. (FIGS. 3A-B) during treatment. From the finite element model of the collagen scaffold, a relationship between lesion area and electric field magnitude is determined by performing numerical integration on the surface of the scaffold for a range of electric field magnitudes (100-3000 V/cm). A curve was constructed and fit to the data using least squares fitting in MATLAB. This resulted in a sixth order polynomial equation. This equation was used to accurately back out the electric field thresholds for each treatment condition without needing to manually determine them using COMSOL. Least squares fitting resulted in a maximum relative error of 4.7%.

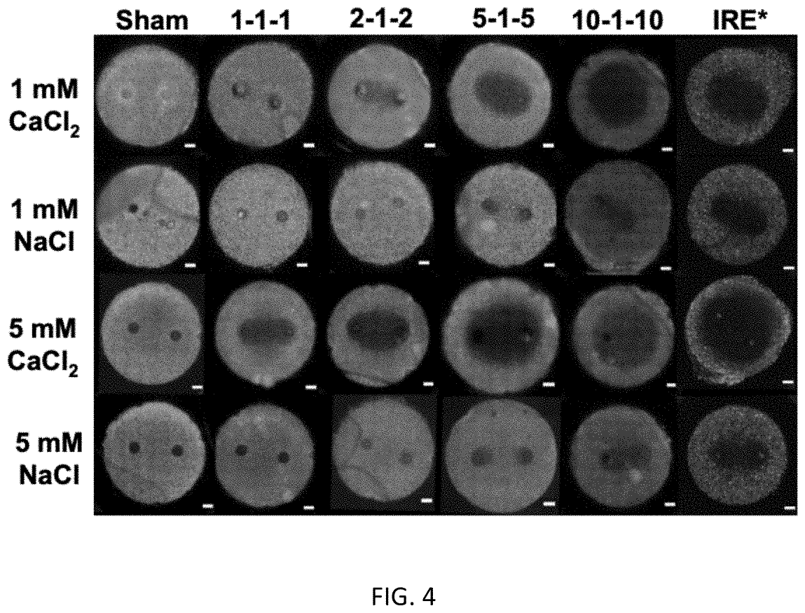

In vitro collagen scaffolds cultured with U251 malignant glioma (MG) cells, treated using H-FIRE with a NaCl control confirms that the field is not high enough to produce a clinically relevant lesion (FIG. 4). Using an applied voltage increased by about 50%, achieves a large ablation volume. Applying a higher energy treatment, however, negates the advantages of H-FIRE because of the risk of introducing deleterious thermal damage as well as increasing the chances of producing muscle contractions, which may in turn increase the possibility of having to administer a neuromuscular block.

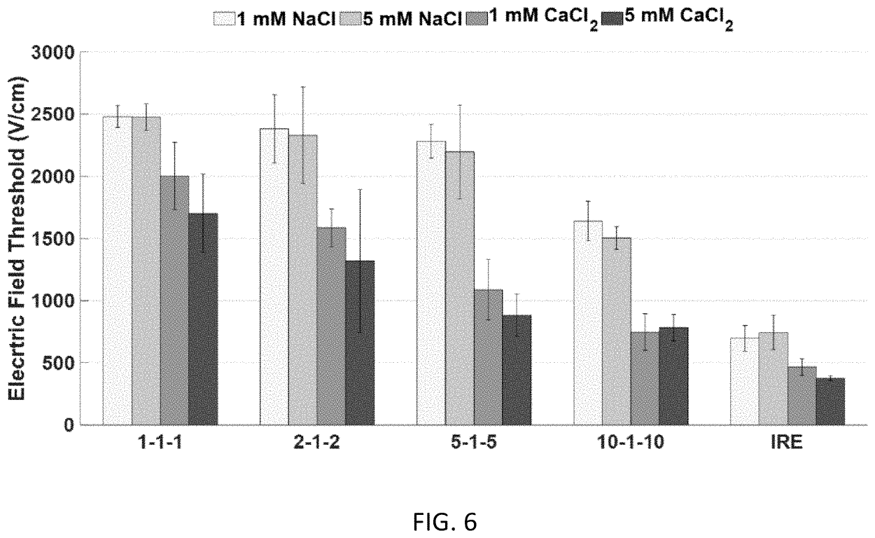

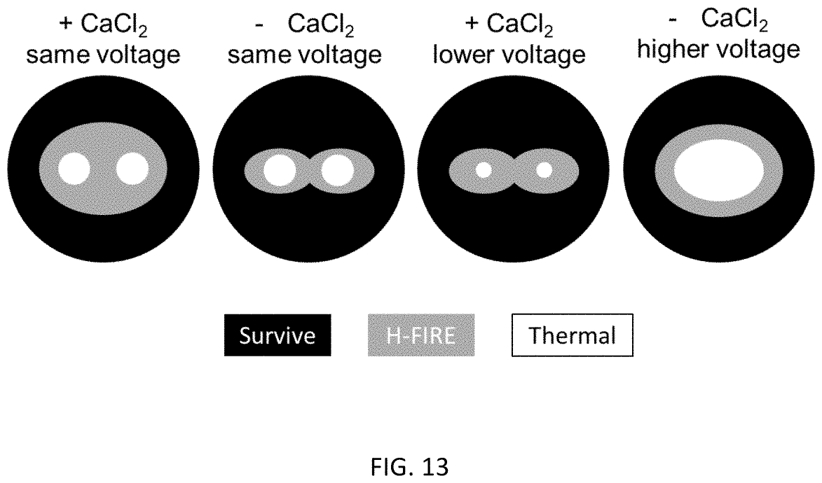

Because H-FIRE treatment results in an irreversibly electroporated zone of a certain size and an associated thermal ablation area within this zone, the inventors have discovered that combining H-FIRE treatment with adjuvant calcium increases/enhances the area and/or margin of ablation without necessitating the use of a higher applied voltage (FIG. 6 and FIG. 13). For example, FIG. 13 shows how the tissue is affected with or without calcium administration and with or without a higher voltage. As illustrated, the region labeled “survive” are cells that are either reversibly electroporated and/or not affected by the electroporation, while the regions labeled “H-FIRE” are irreversibly electroporated in response to H-FIRE protocols and the regions labeled “thermal” are subjected to thermal effects from the electroporation. Additionally, or alternatively, the shape of the area/zone of ablation can also be manipulated and/or controlled with the presence of excess calcium ions during electroporation. For example, different concentrations or amounts of calcium can be administered in different tissue regions for varying levels of effect within the treatment region.

To induce the same area of cell death without calcium, one could increase the applied voltage, which would in turn lead to an increase in joule heating and thermal damage of tissue, due to electrical properties of tissue. This is because the non-thermal mode of cell death does not denature proteins. Using calcium in combination with H-FIRE treatment will allow clinicians to lower the applied voltage by up to 50% or more, therefore lowering thermal damage while maintaining the same ablation area as H-FIRE treatment without calcium. In addition, clinicians may apply the same voltage to increase/enhance the treatment area/zone and/or margin, without an increase in thermal damage. Calcium H-FIRE treatment enhances the IRE to thermal cell death ratio, therefore enhancing the positive immune response seen after treatment. For example, where a clinician would administer H-FIRE without calcium at voltages ranging from 2,500 V to 10,000 V, the H-FIRE voltage could instead be adjusted/lowered 50% or more to below 5,000 V, such as from 1,200 V to 5,000 V when administering calcium in combination with H-FIRE.

In an embodiment of the invention, low voltage H-FIRE treatment is combined with calcium to improve the IRE zone, reduce the size of the thermal ablation zone, and induce an immune response by way of immune cells responding to the tissue injury resulting from the IRE.

In another embodiment of the invention, IRE treatment is combined with calcium to extend the IRE zone while keeping the thermal ablation zone minimal and induce an immune response by way of immune cells responding to the tissue injury. As shown in FIGS. 3A, 3B and 4 these examples have the same thermal zone but the IRE zone is increased with calcium. Therefore, the IRE:thermal ratio and the positive immune response is enhanced.

Accordingly, the inventors have discovered electroporation treatments using lower voltages and/or shorter pulse lengths and adjuvant calcium that are capable of achieving treatment areas/zones and/or margins comparable to that of similar treatments without the added calcium and which can be used to promote a positive immune response. Such protocols include methods of treating tissue comprising: applying a plurality of electrical pulses to a tissue region at a desired voltage and/or with a pulse width of less than 100 μs; and exposing the tissue region to one or more agent; wherein the applying is performed in a manner sufficient to cause electroporation of cells of at least a portion of the tissue region; and wherein the agent is capable of protecting cells from, enhancing or increasing, and/or inhibiting, decreasing, or limiting, one or more effects of the electroporation in at least a portion of the tissue region. In embodiments, the agent administered can comprise calcium to enhance electroporation and/or a non-calcium containing buffer to provide protection to select cells.

These methods can be used to administer any electroporation based therapy, including electroporation, irreversible electroporation, reversible electroporation, electrochemotherapy, electrogenetherapy, supraporation, and/or high frequency irreversible electroporation, or combinations thereof. Of particular interest, such methods can involve performing the applying and the exposing for an increased IRE to thermal cell death ratio in at least a portion of the tissue region, such that a lower thermal effect and an enhanced positive immune response are provided.

Additionally, or alternatively, such methods can involve exposing the tissue region to an agent comprising calcium capable of enhancing one or more of the effects of the electroporation in at least a portion of the tissue region. Additionally, or alternatively, such methods can involve exposing the tissue region to a non-calcium containing buffer capable of limiting one or more of the effects of the electroporation in at least a portion of the tissue region.

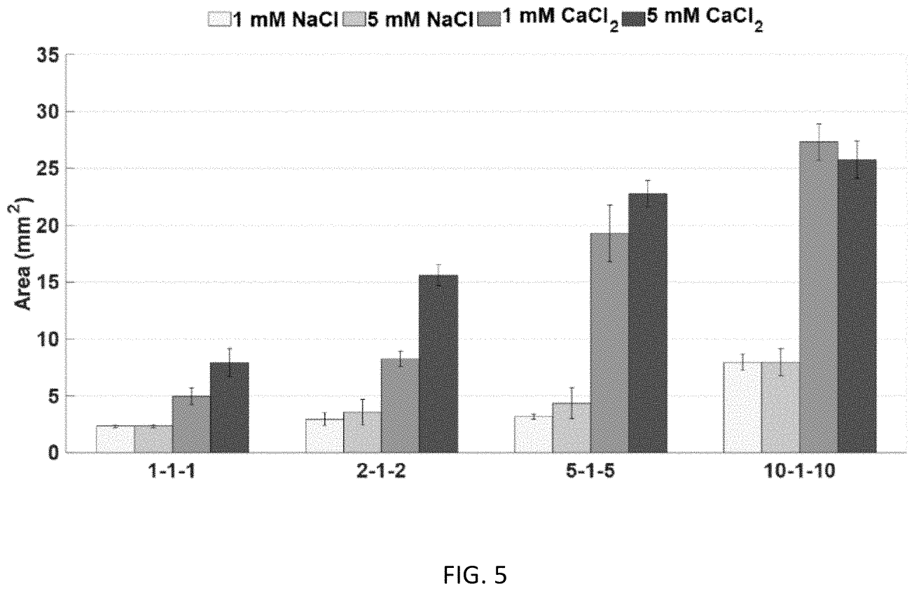

With the addition of CaCl₂, larger ablation volumes are obtained without thermal damage and the need for a neuromuscular block is mitigated. FIG. 4 and FIG. 5 confirm that the addition of 5 mM CaCl₂ with the same applied energy leads to the formation of a lesion that is comparable in size or larger than an IRE treatment without calcium for 2, 5 and 10 μs pulse widths. This discovery is important because it enhances the efficacy of electroporation treatment, such as H-FIRE treatment, producing lesion sizes that are clinically relevant while also alleviating the limitations found with IRE treatment such as muscle contractions.

FIG. 5 quantitatively compares ablation area in response to the different treatments administered. For shorter pulse widths, there is no visible lesion (apart from the electrodes) present for NaCl conditions. This indicates that the cells are reversibly electroporated during H-FIRE treatment and that something more is needed to drive cells to undergo cell death, such as the presence of excess calcium. The increase of ablation area for 1 mM CaCl₂ compared to its NaCl control is nearly 2.3× for the 1-1-1 waveform, 4.4× for the 2-1-2 waveform, 6.1× for the 5-1-5 waveform, and 3.42× for the 10-1-10 waveform. For 5 mM CaCl₂ the increase in ablation area is also apparent. For the 1-1-1 waveform, there is a 3.4× increase, for the 2-1-2 waveform there is a 4.37× increase, for the 5-1-5 waveform there is a 5.24× increase, and for the 10-1-10 waveform there is a 2.66× increase. Thus, ablation sizes for calcium groups for all pulse patterns were statistically significantly larger than that of the NaCl groups. As pulse width increases, the ablation areas for both CaCl₂ concentrations also increases since the cells are permeabilized to a greater extent. The TMP of the cell has been shown to spend more time above the critical threshold as the pulse width increases, therefore resulting in more permeabilization.

From the finite element model, it is possible to determine the electric field threshold required for cell death. FIG. 6 shows that the use of adjuvant calcium reduces the electric field threshold in all experimental conditions. When compared to the NaCl control, 1 mM CaCl₂ reduces the electric field threshold 1.24× for the 1-1-1 waveform, 1.5× for the 2-2-2 waveform, 2.1× for the 5-5-5 waveform, and 2.2× for the 10-1-10 waveform. When comparing 5 mM NaCl to 5 mM CaCl₂, the electric field threshold is reduced 1.46× for the 1-1-1 waveform, 1.77× for the 2-1-2 waveform, 2.49× for the 5-1-5 waveform, and 1.92× for the 10-1-10 waveform. It seems the maximum effect for CaCl₂ is seen with 5 mM CaCl₂ for the 5-1-5 waveform and with 1 mM CaCl₂ for the 10-1-10 waveform.

Although the H-FIRE thresholds are higher than they are for IRE, using calcium significantly lowers the threshold needed to produce a lesion comparable in size to an IRE treatment. Using a 10-1-10 waveform resulted in an electric field threshold (784±107 V/cm) that is comparable to IRE treatment without CaCl₂ (698±103 V/cm) and close to IRE treatment with CaCl₂ (467±67 V/cm). Utilizing 10-1-10 H-FIRE waveforms in combination with adjuvant calcium lowers the required electric field threshold for cell death to a level comparable to IRE treatment. In addition, these waveforms may eliminate the need for custom built generators with complex electronics while avoiding muscle contractions and thermal damage.

For the NaCl controls, the H-FIRE treatment resulted in smaller lesions, again demonstrating that a much higher applied voltage would be needed to produce an ablation without the addition of CaCl₂. The inventors have harnessed this protective function of the non-calcium containing buffer (such as a buffer comprising sucrose, e.g., an NaCl buffer comprising sucrose) as a way of protecting cells present in one or more non-target treatment zones during treatment.

Accordingly, additional methods of selectively treating cells are included that involve applying a plurality of electrical pulses to a tissue region; and exposing the tissue region to one or more agent; wherein the applying of the electrical pulses is performed in a manner sufficient to treat cells of the tissue region with high-frequency irreversible electroporation (H-FIRE); and wherein the agent is capable of protecting cells from, enhancing or increasing, and/or inhibiting, decreasing, or limiting, one or more effects of the H-FIRE and that involve exposing a first tissue region to an agent comprising calcium in an amount sufficient to enhance one or more effects of the H-FIRE; and exposing a second tissue region to a non-calcium containing buffer in an amount sufficient to limit one or more effects of the H-FIRE. Such methods can provide for selective treatment of tissue/cells by administering one type of treatment in a first region and a second type of treatment in a second region. In embodiments, selectively treating cells/tissue according to the invention can be used to treat cancer cells in a first tissue region and non-cancerous and/or non-target cells in a second tissue region.

To investigate how CaCl₂ and NaCl treatments affect permeabilization of the cells, the areas for reversible electroporation and their corresponding reversible electric field thresholds were characterized. Reversible electroporation thresholds for H-FIRE treatment have not been extensively characterized, making this work one of the first to quantify reversible thresholds for a range of unexplored pulse durations.

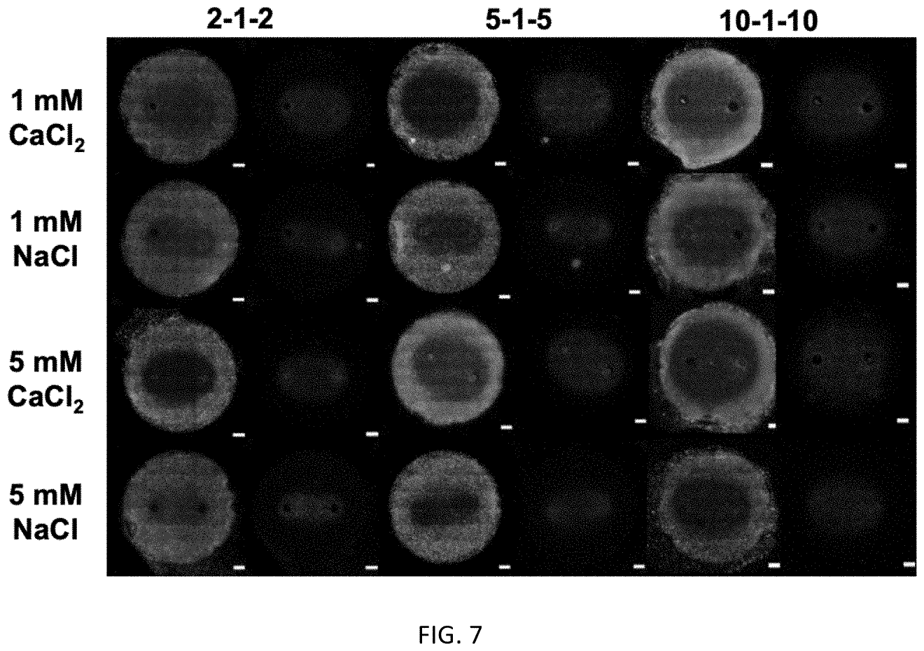

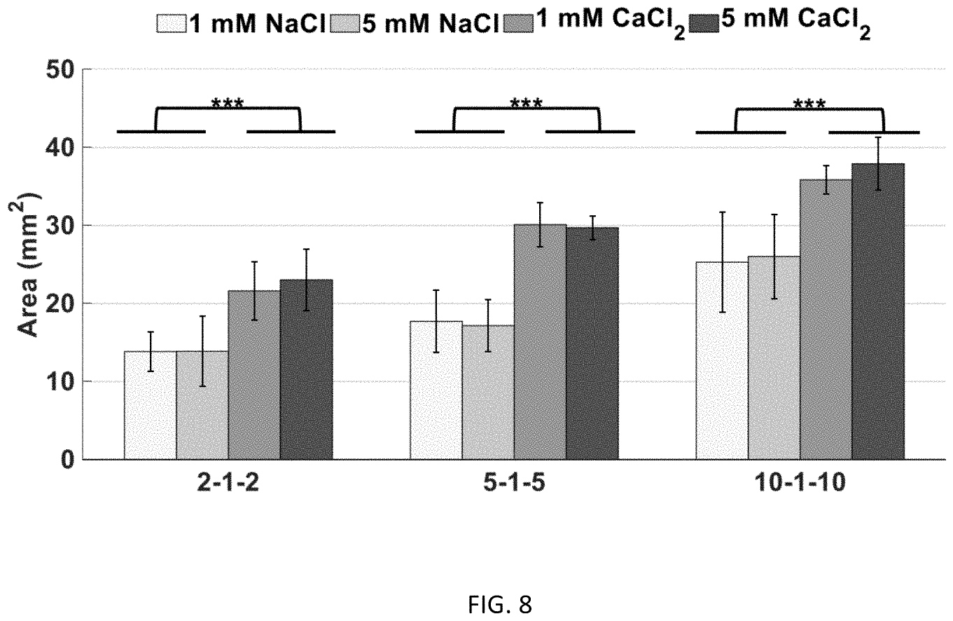

FIGS. 7-8 show that both NaCl solutions had smaller reversible areas (i.e., the darker interior regions of the images) than their CaCl₂ counterparts, therefore the buffers used seemed to affect the extent that the cells were being permeabilized. In addition, it appeared that the difference between NaCl and CaCl₂ reversibly electroporated zones was similar for all waveforms. To compare the reversible zones more quantitatively, area was measured using the custom MATLAB algorithm.

FIG. 8 shows that for 1 mM CaCl₂, the reversible zone was 1.56× larger than the 1 mM NaCl zone for the 2-1-2 waveform, 1.7× larger for the 5-1-5 waveform, and 1.42× larger for the 10-1-10 waveform. For 5 mM CaCl₂, the reversible zone was 1.66× larger than the 1 mM NaCl zone for the 2-1-2 waveform, 1.73× for the 5-1-5 waveform, and 1.46× larger for the 10-1-10 waveform. There was no statistically significant difference between 1 mM and 5 mM treatments for the reversible case, although, after 24 h, the 5 mM CaCl₂ areas are larger than the 1 mM CaCl₂ for shorter pulse durations suggesting that cells exposed to lower levels of calcium recover to some extent.

Thus, the inventors have discovered that the presence of excess or added calcium in combination with H-FIRE can provide not only for an enhanced irreversible electroporation zone but also can provide for an enhanced surrounding reversible electroporation zone. Accordingly, additional methods of selectively treating cells are included that involve applying a plurality of electrical pulses to a tissue region; and exposing the tissue region to one or more agent comprising calcium; wherein the applying of the electrical pulses is performed in a manner sufficient to treat cells of the tissue region with high-frequency irreversible electroporation (H-FIRE); and wherein the agent is capable of protecting cells from, enhancing or increasing, and/or inhibiting, decreasing, or limiting, one or more effects of the H-FIRE, such as providing for an enhanced RE zone. Such methods are useful for administering IRE to undesirable tissue (e.g., tumors) in a first zone, while also increasing the amount of RE in a second zone for either an enhanced immune response and/or to provide for a better opportunity for the administration of other agents such as chemotherapy agents or gene/DNA delivery in the RE zone, both of which could be used to complement/enhance the IRE treatment.

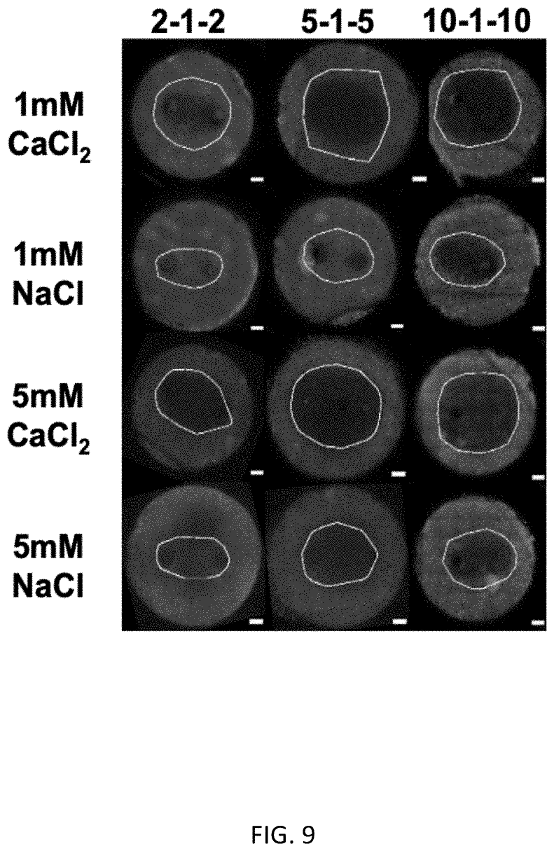

For example, FIG. 9 shows that reversible electroporation zones (white circled regions) were larger than irreversible zones (darker interior regions) for all conditions tested. For the 2-1-2 waveform, reversible zones were 4.68× larger than irreversible zones for 1 mM NaCl, 3.88× for 5 mM NaCl, 2.62× for 1 mM CaCl₂, and 1.47× for 5 mM CaCl₂. For the 5-1-5 waveform reversible zones were 5.58× larger than irreversible zones for 1 mM NaCl, 3.94× for 5 mM NaCl, 1.67× for 1 mM CaCl₂, and 1.3× for 5 mM CaCl₂. For the 10-1-10 waveform, reversible zones were 3.17× larger for 1 mM NaCl, 2.94× larger for 5 mM NaCl, 1.32× larger for 1 mM CaCl₂, and 1.59× larger for 5 mM CaCl₂ when compared to irreversible zones. It is noted that in FIG. 9 there are some dark areas beyond the RE zone circled in white because sometimes there are bubbles of media that block the light from the microscope. Cells that are dead will be stained with propidium iodide which is darker in color here. It is not possible to have both the reversible and irreversible zones characterized on the same hydrogel because it requires different procedures, therefore, the RE zones overlaid here are zones that best represent the mean area that was measured.

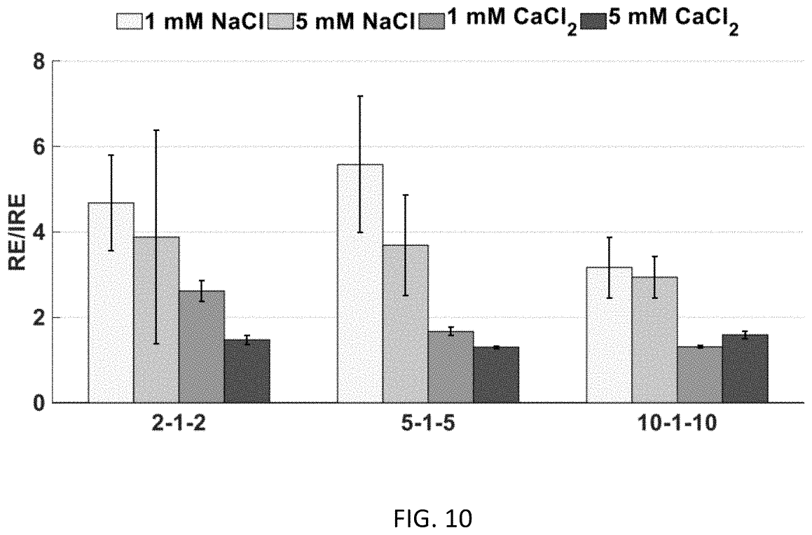

In FIG. 10, the difference between reversible and irreversible electroporation areas was largest for NaCl solutions. When CaCl₂ was used, the difference between reversible and irreversible electroporation areas decreased because the cells that were reversibly electroporated are driven instead to undergo cell death (IRE) due to uptake of excess calcium. Generally, as pulse duration increased, the difference between reversible and irreversible electroporation areas also decreased.

Using the finite element model of H-FIRE treatment in the scaffold, the corresponding electric field thresholds for each ablation area were characterized. Table 2 shows that using adjuvant calcium reduces the electric field threshold (compared to the controls) in all experimental conditions.

Boxes highlight that 10-1-10 H-FIRE treatment with calcium results in comparable electric field thresholds to standard IRE treatment. When compared to the NaCl control, 1 mM CaCl₂ reduces the electric field threshold 1.24× for the 1-1-1 waveform, 1.48× for the 2-1-2 waveform, 2.05× for the 5-1-5 waveform, and 2.19× for the 10-1-10 waveform. When comparing 5 mM NaCl to 5 mM CaCl₂, the electric field threshold is reduced 1.46× for the 1-1-1 waveform, 1.91× for the 2-1-2 waveform, 2.43× for the 5-1-5 waveform, and 1.83× for the 10-1-10 waveform. It seems the maximum effect for CaCl₂ is seen with 5 mM CaCl₂ for the 5-1-5 waveform and with 1 mM CaCl₂ for the 10-1-10 waveform. Using a 10-1-10 waveform with 1 mM CaCl₂ results in an electric field threshold of 771±129 V/cm, reducing the threshold to less than half its value with NaCl (1641±159 V/cm). It is important to note that 1 mM CaCl₂ also reduces the threshold to a level that is comparable to an IRE treatment with NaCl (698±103 V/cm).

It should be noted that despite most tissues having extracellular calcium concentrations around 1 mM, calcium ions are often bound by other macromolecules and only a small fraction are free in the extracellular fluid. Therefore, when treating tumors in vivo using calcium electroporation, administration of exogenous calcium is likely needed to provide the desired effect. Ensuring a desired distribution of calcium in the tissue region (e.g., uniform distribution) to be treated may be difficult to achieve due to one or more of leaky vasculature, high interstitial pressure and convective forces that drive fluid out of the tumor, however, one remedy could be to co-administer the adjuvant and/or buffer during electroporation, and/or administer immediately prior to treatment, and/or in addition to (or alternatively to) administering prior to treatment. Embodiments can include injecting calcium, such as calcium chloride, directly into a tumor and delivering electroporation, such as H-FIRE pulses, through electrodes inserted into the tumor. For protecting or sparing tissue, sucrose buffer could be injected into blood vessels near tumors prior to treatment to prevent electroporation of the endothelial cells in the vessel. Electrodes may be designed to inject CaCl₂ into the tumor during treatment while also delivering a non-calcium buffer (such as a buffer comprising sucrose, e.g., an NaCl buffer comprising sucrose) on the tumor borders to protect surrounding tissue. Venofer is an Iron Sucrose solution (300 mg/ml sucrose w/v) that is administered intravenously and utilized to treat anemia. Such solutions could also be used to protect certain tissues from effects of electroporation. Most side effects are associated with the speed of administration and include dizziness, nausea, vomiting, and muscle cramps. Indeed, any composition comprising sucrose can be used as the non-calcium containing agent for protecting cells against one or more effects of electroporation.

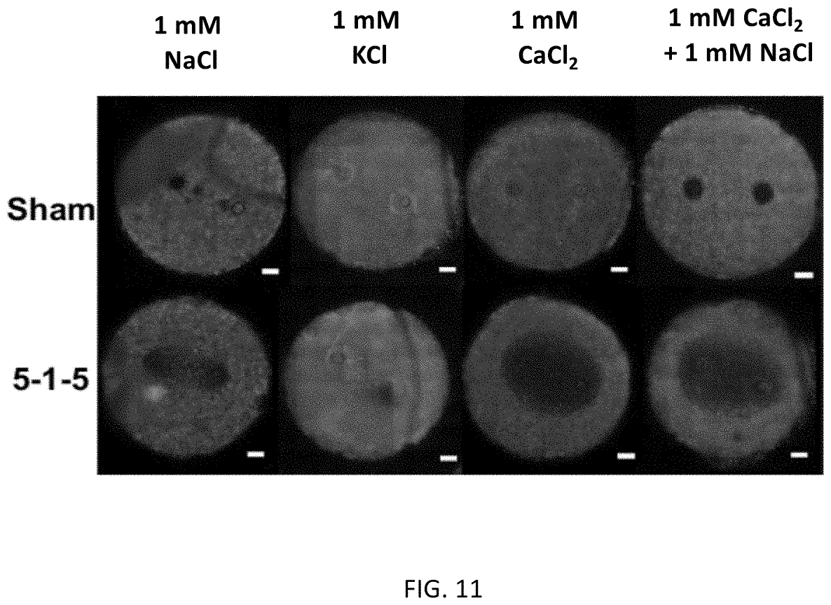

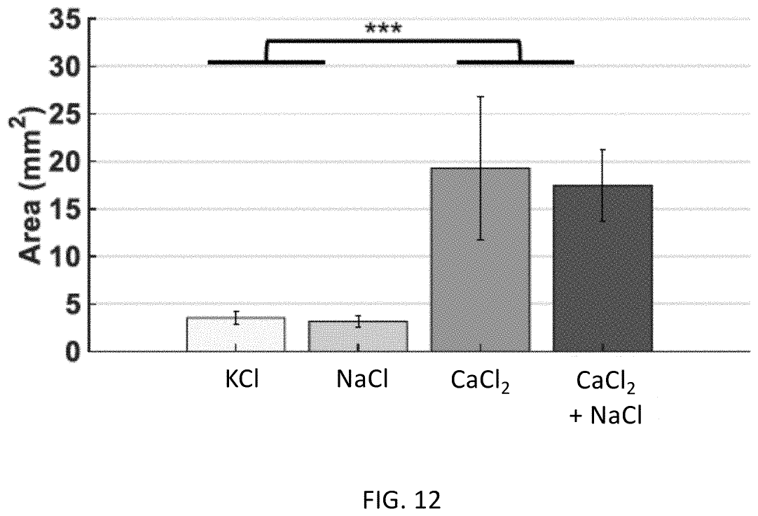

There are several ion channels in the plasma membrane that act to pump calcium out of the cell. One of these pumps is the Na⁺-Ca²⁺ exchanger. The exchanger works to allow Na⁺ to be transported into the cell while pumping Ca²⁺ out of the cell. To investigate whether NaCl would be able to aid the cells in pumping the excess Ca²⁺ out, a solution that contained 1 mM of both ions was tested. A solution of KCl (potassium chloride) was also tested to determine whether the enhanced cell death effect is unique to calcium. FIGS. 11-12 show that the CaCl₂ solution and the combined solution of NaCl and CaCl₂ did not result in significantly different ablation sizes (p<0.001), therefore, NaCl does not rescue the cells from the effect of excess Ca²⁺. In addition, testing a 1 mM KCl solution did not result in statistically significant ablation size relative to the NaCl control, again confirming that the enhanced ablation areas were unique to the presence of excess calcium ions.

The inventors have thus discovered and provide a method of selectively treating cells, comprising: applying a plurality of electrical pulses to first and second tissue regions; exposing the first tissue region to a first agent in a manner such that more cell death occurs within the first tissue region than without presence of the first agent; and exposing the second tissue region to a second agent in a manner such that: less cell death, or no cell death, occurs within the second tissue region than without presence of the second agent; and/or the second tissue region comprises a zone of reversible electroporation, the zone being enhanced by presence of the second agent.

In such embodiments, the plurality of electrical pulses the first and/or second tissue region are capable of electroporation based therapy, electroporation, irreversible electroporation, reversible electroporation, electrochemotherapy, electrogenetherapy, supraporation, and/or high frequency irreversible electroporation, or combinations thereof.

For example, ablation of tissue can be performed in the first tissue region by administering IRE or H-FIRE along with an adjuvant comprising calcium. Addition of the calcium can be in an amount sufficient to increase/enhance the treatment area/zone and/or margin and result in a larger ablation volume in the first tissue region. In embodiments, CaCl₂ is used. The calcium adjuvant can be administered before and/or during the electroporation treatment, such as by injection into the tissue region as discussed above and/or in the amounts/concentrations provided.

A concurrent part of the treatment in selectively treating cells according to this embodiment, entails administering the same or similar or a different type of electroporation treatment in the second tissue region. A non-calcium buffer is administered into the second tissue region to protect the cells in that region from ablation, or limit the amount of ablation. In embodiments, a buffer comprising sucrose, e.g., an NaCl buffer comprising sucrose, is used as the non-calcium containing buffer. Thus, using calcium adjuvant in the first tissue region with a selected electroporation modality, a desired ablation volume can be achieved, while using the non-calcium buffer in the second tissue region, a protective effect such as less or no cell death will occur in the second tissue region.

Such selective ablation techniques are useful for achieving an increased IRE to thermal cell death ratio in the first tissue region, such that a lower thermal effect and an enhanced positive immune response are provided. Additionally, or alternatively, cells in the second tissue region can be spared, which is useful in the context of preserving vasculature, nerve tissue, and/or tissue near the vasculature or the nerve tissue and/or tissue near one or more electrodes used in applying the plurality of electrical pulses, especially from Joule heating.

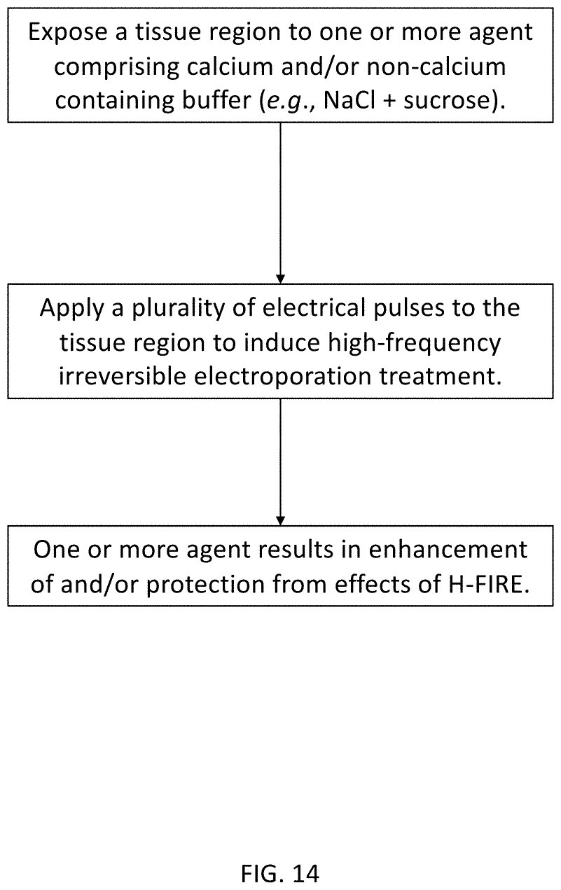

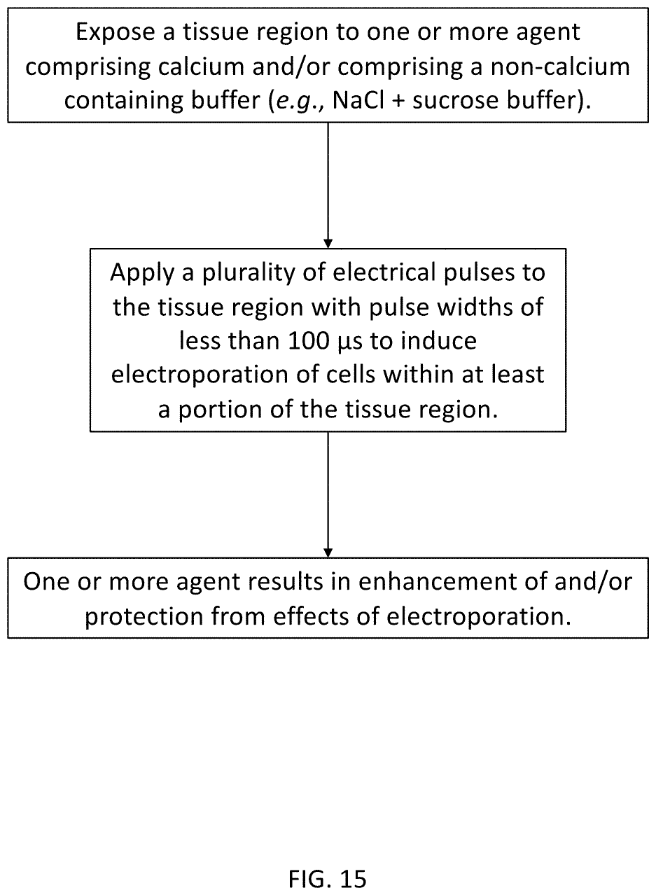

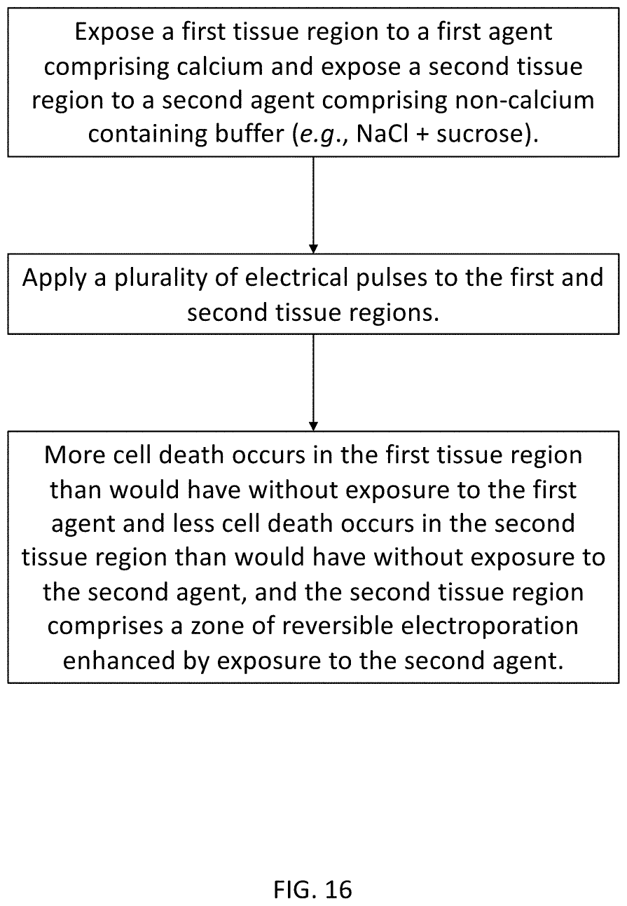

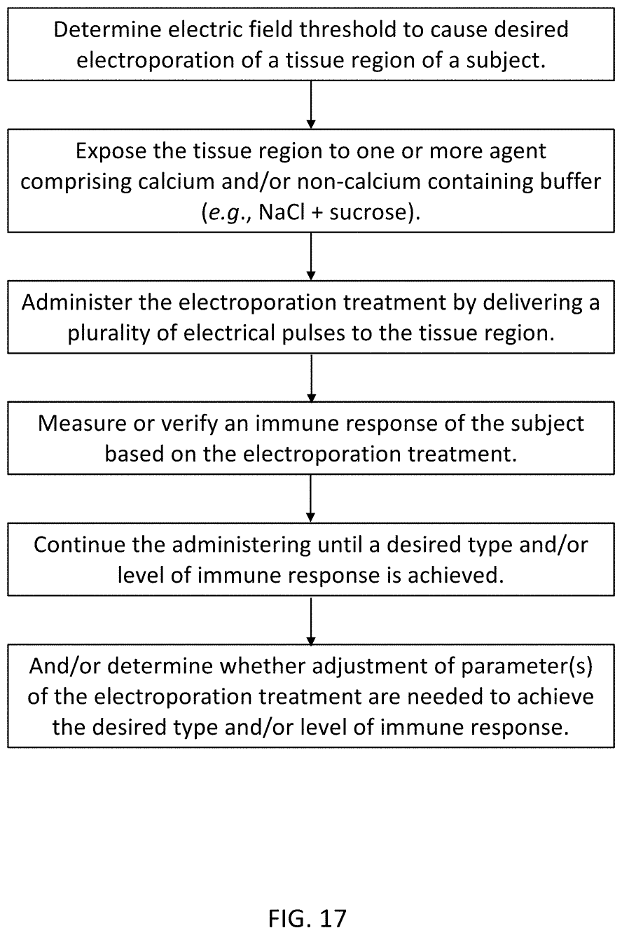

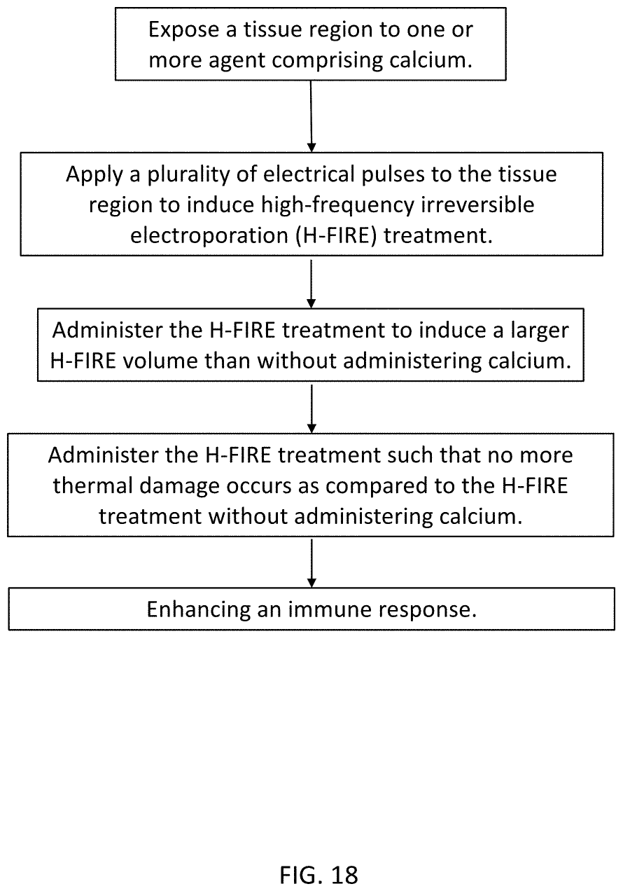

Additionally, various exemplary method embodiments of the invention are illustrated in FIGS. 14-18. According to embodiments, one or more features of the methods described in this specification and corresponding figures can be used to achieve one or more goals outlined herein.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of treating tissue comprising:
applying a plurality of electrical pulses to a tissue region; and
exposing the tissue region to one or more agent;
wherein the applying of the electrical pulses is performed in a manner sufficient to treat cells of the tissue region with high-frequency irreversible electroporation (H-FIRE); and
wherein the agent is capable of protecting cells from, enhancing or increasing, and/or inhibiting, decreasing, or limiting, one or more effects of the H-FIRE.

2. The method of claim 1, wherein one or more of the effects of the H-FIRE is chosen from cell death; quick cell death on the order of seconds or minutes; slow cell death on the order of hours, days, weeks, months or years; apoptosis; necrosis; heat; thermal effects; cell membrane permeabilization; inflammatory response; blood brain barrier disruption; transient blood brain barrier disruption; permanent damage; transient damage; immune response; a reversible electroporation zone and combinations thereof.

3. The method of claim 1, wherein the agent comprises calcium or a non-calcium buffer.

4. The method of claim 1, wherein the agent comprises calcium in an amount sufficient to:
increase an area of ablation to a size comparable to that expected from H-FIRE administered using a higher voltage; and/or
provide an increased IRE to thermal cell death ratio than without the calcium, such that a lower thermal effect and an enhanced positive immune response are provided.

5. The method of claim 4, wherein the positive immune response is promoted by immune cells present beyond the tissue region to which the plurality of electrical pulses are applied.

6. The method of claim 1, wherein one or more of the agents comprises calcium in an amount capable of increasing one or more effects of the H-FIRE in the tissue region.

7. The method of claim 1, wherein one or more of the agents comprises sucrose capable of protecting cells from, inhibiting, decreasing, or limiting one or more effects of the H-FIRE.

8. The method of claim 1, wherein the exposing of the tissue region to the agent comprises:
exposing a first tissue region to an agent comprising calcium in an amount sufficient to enhance one or more effects of the H-FIRE; and
exposing a second tissue region to a non-calcium containing buffer in an amount sufficient to limit one or more effects of the H-FIRE.

9. The method of claim 8, wherein the first tissue region comprises cancer cells.

10. The method of claim 9, wherein the second tissue region comprises non-cancerous cells.

11. A method of treating tissue comprising:
applying a plurality of electrical pulses to a tissue region;
wherein the electrical pulses have a pulse width of less than 100 μs; and
exposing the tissue region to one or more agent;
wherein the applying is performed in a manner sufficient to cause electroporation of cells of at least a portion of the tissue region; and
wherein the agent is capable of protecting cells from, enhancing or increasing, and/or inhibiting, decreasing, or limiting, one or more effects of the electroporation in at least a portion of the tissue region.

12. The method of claim 11, wherein the agent comprises calcium or a non-calcium containing buffer.

13. The method of claim 11, wherein the plurality of electrical pulses are capable of electroporation based therapy, electroporation, irreversible electroporation, reversible electroporation, electrochemotherapy, electrogenetherapy, supraporation, and/or high frequency irreversible electroporation, or combinations thereof.

14. The method of claim 11, wherein the applying and the exposing together provide for an increased IRE to thermal cell death ratio in at least a portion of the tissue region, such that a lower thermal effect and an enhanced positive immune response are provided.

15. The method of claim 11, wherein the exposing of the tissue region to the agent comprises exposing the tissue region to an agent comprising calcium capable of enhancing one or more of the effects of the electroporation in at least a portion of the tissue region.

16. The method of claim 11, wherein the exposing of the tissue region to the agent comprises exposing the tissue region to a non-calcium containing buffer capable of limiting one or more of the effects of the electroporation in at least a portion of the tissue region.

17. A method of selectively treating cells, comprising:
applying a plurality of electrical pulses to first and second tissue regions;
exposing the first tissue region to a first agent in a manner such that more cell death occurs within the first tissue region than without presence of the first agent; and
exposing the second tissue region to a second agent in a manner such that:
less cell death, or no cell death, occurs within the second tissue region than without presence of the second agent; and/or
the second tissue region comprises a zone of reversible electroporation, the zone being enhanced by presence of the second agent.

18. The method of claim 17, wherein the first agent comprises calcium and the second agent comprises a non-calcium buffer.

19. The method of claim 17, wherein the applying and the exposing together provide for an increased IRE to thermal cell death ratio, such that a lower thermal effect and an enhanced positive immune response are provided.

20. The method of claim 17, wherein the second tissue region comprises:
vasculature, nerve tissue, and/or tissue near the vasculature or the nerve tissue; and/or tissue near one or more electrodes used in applying the plurality of electrical pulses.
